SEQUENCE LISTING (1 sequence)

SEQ ID NO: 1 - protein, 320 aa, Dermatophagoides pteronyssinus
  SIGNAL (1)..(18)
  PROPEP (19)..(98)
  mat_peptide (99)..(320)
MKIVLAIASLLALSAVYARPSSIKTFEEYKKAFNKSYATFEDEEAARKNFLESVKYVQSNGGAINHLSDLSLDEFKNRFLMSAEAFEHLKTQFDLNAETNACSINGNAPAEIDLRQMRTVTPIRMQGGCGSCWAFSGVAATESAYLAYRNQSLDLAEQELVDCASQHGCHGDTIPRGIEYIQHNGVVQESYYRYVAREQSCRRPNAQRFGISNYCQIYPPNVNKIREALAQTHSAIAVIIGIKDLDAFRHYDGRTIIQRDNGYQPNYHAVNIVGYSNAQGVDYWIVRNSWDTNWGDNGYGYFAANIDLMMIEEYPYVVIL

(19) United States
(12) Patent Application Publication
(10) Pub. No.: US 2008/0254041 A1
(43) Pub. Date: Oct. 16, 2008

(54) Method for Selecting an Immunotherapeutic Preparation

(75) Inventors: Steffen Ernst, Bronshoj (DK); Charlotte Georgi Jakobsen, Copenhagen N (DK); Erwin Ludo Roggen, Lyngby (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 10/571,496

(22) PCT Filed: Sep. 10, 2004
(86) PCT No.: PCT/DK2004/000598

§ 371 Date: Apr. 7, 2006

(30) Foreign Application Priority Data
Sep. 11, 2003 (DK) PA 2003 1311

(51) Int. Cl. A61K 39/395

(52) U.S. Cl. 424/171.1; 424/130.1

(57) ABSTRACT

The present invention relates to a method for selecting one or more immunotherapeutic preparation having improved properties as a vaccine against a target antigen, said method comprising raising antibodies against a group of candidate preparations and selecting one or more preparations which produces antibodies having higher affinity against the target antigen than does anti-bodies raised against at least one other preparation from the group. More particularly it relates to the need to provide recombinant allergens (Bet V 1) for SIT which have been modified by protein engineering.

### FIELD OF INVENTION

The present invention relates to a method for identifying and selecting an immunotherapeutic preparation having improved properties as a vaccine against a target antigen as well as a method for selecting an improved immunotherapeutic administration regime for a vaccine against a target antigen. Also included are variant antigens obtainable by the method of the invention as well as the use of such variant antigens in a vaccine.

### BACKGROUND OF THE INVENTION

Allergens present in the environment or in occupational situations, can induce immunological responses, such as an atopic allergic response, in susceptible individuals among humans and animals. Allergic responses may range from hay fever, rhinoconjunctivitis, rhinitis, and asthma, and in cases when the sensitized individual is exposed, e.g., to bee sting or insect bites, even to systemic anaphylaxis and death.

An individual may become sensitized to such polypeptides, termed allergens, by inhalation, direct contact with skin or eyes, ingestion or injection. The general mechanism behind an allergic response is divided into a sensitization phase and a symptomatic phase. The sensitization phase involves a first exposure of an individual to an allergen. This event activates specific T- and B-lymphocytes, and leads to the production of allergen specific antibodies, such as immunoglobulin E (IgE). The specific IgE antibodies bind to IgE receptors on mast cells and basophils, among others, and the symptomatic phase is initiated upon a second exposure to the same or a homologous allergen. The allergen will bind to the cell-bound IgE, and the polyclonal nature of the antibodies results in bridging and clustering of the IgE receptors, and subsequently in the activation of mast cells and basophils. This activation results in the release of various chemical mediators, such as histamine, heparin, proteases, prostaglandin D2 and leukotrienes, involved in the early as well as late phase reactions of the symptomatic phase of allergy.

For certain forms of IgE-mediated allergies, a therapy exists, called specific immune therapy (SIT), or specific allergy vaccination (SAV) or immunotherapy (IT), which comprises repeated parenteral or mucosal (e.g., sublingual) administration of allergen preparations formulated as a vaccine (Int. Arch. Allergy Immunol., 1999, vol. 119, pp 1-5). This leads to reduction of the allergic symptoms, most likely due to induction of a protective, non IgE-based immune response, possibly by modulation of the existing Th2 response and/or a redirection of the immune response towards the immunoprotective (Th1) pathway (Int. Arch. Allergy Immunol., 1999, vol. 119, pp 1-5).

The status and prospects of SIT has recently been reviewed (Frew, J Allergy Clin Immunol. Vol. 111, p. S721-719, 2003; Bosquet, 2000, BioDrugs, vol. 13, p. 61-75; Valenta, 2003, Nature Rev. Immunology, vol. 2, pp. 446-453, 2003). SIT is known to improve clinical performance of patients. However, SIT also involves risk of serious side-reactions: for example, 26 deaths have been registered in UK from 1957-1986 due to SIT (Frew, J Allergy Clin Immunol. Vol. 111, p. S721-719, 2003). As SIT is also a long and cumbersome process for the patient, it is desired to find allergy vaccines with increased vaccination potency, in order to allow simpler, shorter, and/or lower-dosage (which would be safer) administration regimes. As the mechanism of clinical protection is still under investigation and debate in the literature, it is currently not clear which characteristics of the immune response to allergen vaccination that should be sought after, when selecting among various vaccine compositions.

SIT is known to induce increased levels of IgG (including IgG4) (Witteman, 1996, Int. Arch Allergy Immunol, 109 p. 369). However, there is a poor correlation between the amount of specific IgG and clinical protection. (Frew, J Allergy Clin Immunol. Vol. 111, p. S721-719, 2003).

A possible clue to a causative effect of SIT-induced IgG is given by van Neerven, who found that allergen presentation (and subsequent C D4+T-cell activation) was facilitated by patient allergen-specific serum IgE, and that this facilitation is inhibited by IgG from patients receiving SIT. The authors speculate that SIT-induced increased levels of IgG and especially IgG4 are responsible for this, however, they note that patients with short history of SIT and high levels of specific IgG4 were less effective in inhibiting the reaction than patients with long history of SIT and lower levels of specific IgG4, hence ‘strongly indicating that the inhibition of serum facilitated allergen presentation cannot solely be explained by increased serum-levels of birch allergen-specific IgG4’(van Neerven, 1999, J. Immunology, 163, pp 2944-2952).

Hence, there exist in the literature at least two proposed mechanisms of antibody mediated protective effect of SIT: a) direct competition with IgE for allergen binding, leading to reduced productive IgE-Fc (epsilon) RI activation on effector cells (such as mast cells) and b) competition with IgE for allergen-binding and CD23-mediated allergen presentation on B-cells leading to reduced T-cell response upon local exposure to allergen.

In another study, Jacobsen et al.; Allergy, vol 52, pp. 914-920, 1997, followed a cohort of patients for six years after they received SIT with tree pollen extracts. They used inhibition of binding of Iodine-125-labelled birch pollen allergen (Bet v 1) to a number of solid-phase bound monoclonal antibodies by patient serum samples depleted of IgG. This presumably gave a measure for the affinity of allergen to the patient serum antibodies. It was concluded that ‘the affinity of the IgE antibodies remained unaffected . . . [by SIT]’). This finding is supported recently by Svensson et al.(Molecular Immunology vol. 39, pp. 603-612, 2003), who find that ‘the binding avidities changed less than 20% pre- and post SIT’, which is taken as evidence that no binding affinity or avidity changes occur during immunotherapy.

In general, there is a need to provide recombinant allergens for SIT which have been modified by protein engineering. Most groups working in this field is targeting a reduction of IgE based responses in order to reduce these an create a safer vaccine molecule (see e.g. Swoboda, Eur. J. Immunol, vol. 32, pp. 270-280, 2002 or WO02/40676). These studies do not mention the prospects of using protein engineering as a tool to generate more efficacious vaccine molecules.

### SUMMARY OF THE INVENTION

The present invention solves the problem of high clinical symptom scores among subjects suffering allergic disease, even after receiving conventional immunotherapeutic vaccine preparations, by providing recombinant variant antigens modified by protein engineering.

In a first aspect the present invention relates to a method for selecting one or more immunotherapeutic preparation having improved properties as a vaccine against a target antigen, said method comprising raising antibodies against a group of candidate preparations and selecting one or more preparations which produces antibodies having higher affinity against the target antigen than antibodies raised against at least one other preparation from the group.

In a second aspect the present invention relates to A method for selecting one or more improved immunotherapeutic administration regimes for a vaccine against a target antigen, said method comprising raising antibodies against the vaccine through a group of candidate administration regimes and selecting one or more administration regimes having higher affinity against the target antigen than antibodies raised against at least one other administration regime from the group.

In a third aspect the present invention relates to an antigen obtainable by the method of the invention.

In a fourth aspect the present invention relates to the use of antibody affinity towards a target antigen for selecting one or more immunotherapeutic preparation, comprising a vaccination antigen, having improved properties as a vaccine against the target antigen.

### BRIEF DESCRIPTION OF DRAWINGS

FIG. 1

Typical sensorgram showing different antibody samples, purified from serum from patient number 4, binding to Bet v 1; serum, total IgG, total IgE, Bet v 1-specific IgG1, and Bet v 1-specific IgG4. RU: resonance units.

FIG. 2

Binding affinities of Bet v 1 specific IgE, -IgG1, and -IgG4 for each individual patient. Patients are ranked after descending Kₐ values for binding to IgE, and after SIT and no SIT.

### DETAILED DESCRIPTION OF THE INVENTION

### Definitions

A “target antigen” is defined as a polypeptide or protein, towards which the patient is intended to become less sensitive to when encountering it in the environment or in an occupational setting subsequent to vaccination.

A “vaccination antigen” is defined as a polypeptide or protein used for vaccination; e.g. the target antigen or a variant thereof. A “candidate vaccination antigen” or “potential vaccination antigen” is used in the usual meaning of “candidate” or “potential”, for example to describe a polypeptide or protein that is evaluated with the purpose of selecting a vaccination antigen to be tested in animals, or in another example to designate a group of polypeptides or proteins, from which one vaccination antigen was chosen by the methods of the patent.

If the target antigen is an allergen, it may be called a target allergen. When the vaccination antigen is an allergen, it may be called a vaccination allergen.

An “immunotherapeutic preparation” is meant to at least include a vaccination antigen.

“Environmental allergens” are protein allergens that are present naturally. They include pollen, dust mite allergens, pet allergens, food allergens, venoms, etc.

“Commercial allergens” are protein allergens that are being manufactured or extracted for use by humans. They include industrial enzymes and microorganisms, pharmaceutical proteins, antimicrobial peptides, as well as allergens of transgenic plants.

An “epitope” or a “B-cell epitope”, as used in this context, is an antigenic determinant and the structural area on a complex antigen that can combine with or bind an antibody. It can be discontinuous in nature, but will in general have a size of 1 kD or less (about 10 amino acids or less). The size may be 3 to 10 amino acids or 5 to 10 amino acids or even 7 to 10 amino acids, depending on the epitope and the polypeptide.

The term “epitope pattern” as used herein is to be understood as a consensus sequence of antibody binding peptides. An example is the epitope pattern A R R*R. The sign “*” in this notation indicates that the aligned antibody binding peptides included a non-consensus moiety between the second and the third arginine. That moiety may be any amino acid or a few amino acids or no amino acid. Epitope patterns are used to identify epitopes and minimal epitopes on complex antigens.

The term “anchor amino acid” as used herein is to be understood as conserved individual amino acids of an epitope pattern recurring in all peptides bound by monospecific antibodies used to define that pattern. Anchor amino acid will usually also be the amino acid of a minimal epitope on the full polypeptide.

The “antigenicity” of a polypeptide indicates, in this context, its ability to bind antibodies e.g., of IgE and/or IgG and/or other immunoglobulin classes.

The “IgE-antigenicity” of a polypeptide as used herein, indicates its ability to bind IgE antibodies.

The “immunogenicity” of a polypeptide indicates its ability to stimulate antibody production and immunological reactions in exposed animals, including humans.

The “allergenicity” of a polypeptide indicates its ability to stimulate IgE antibody production and allergic sensitization in exposed animals, including humans.

The term “affinity” as used in this context indicates the conventional bimolecular binding affinity as well as the apparent affinity of interaction between a monomolecular species and a polymolecular species (e.g. a polyclonal antibody).

The “apparent affinity” between a monomolecular species (e.g. an antigen) and a polymolecular species (e.g. a polyclonal antibody) is defined as the affinity constant found when data from binding interaction studies are analysed as if there were only two species present.

A “protein variant” is a protein, that has been modified from another protein (e.g. a wild-type protein), e.g. by genetic engineering of the gene, such that the resulting protein variant has another amino acid sequence than the original protein.

### Method for Selecting Immunotherapeutic Preparations

The methods of the present invention relies on affinity of antibodies towards a target antigen to identify and select improved vaccine preparations or administration regimes. The invention encompasses selection of preparations or administration regimes among groups of such which produces antibodies having affinities against a target antigen which are higher than for other candidate of the group, thus having improved properties over other candidates members of the group. Accordingly, selected preparations or administration regimes according to the invention do not have to the best among the group, as long as they are not the worst. It is however preferred that selected preparations or administration regimes are those in the group of candidates producing antibodies having the highest affinity towards the target antigen. In other preferred embodiments selected preparations or administration regimes are those corresponding the 2'nd to 10'th highest affinity.

The present invention relates to the construction of more efficient vaccine antigens, in particular more effective allergy vaccine antigens and to a method for identifying and selecting such improved antigens.

Upon measurement of antibody binding properties of serum from subjects suffering from birch pollen allergy which have been subjected to Specific Immune Therapy (SIT) with birch pollen extracts, such measurements surprisingly show that their clinical symptom score is inversely related to the affinity of their specific IgG1 and even more pronounced of specific IgG4 for binding to bet v1 allergen. There was no correlation between clinical symptoms and serum levels of specific IgE, IgG1 or IgG4, nor were there correlation to the affinity of specific IgE.

This has led to the surprising finding that a vaccination allergen for SIT will be more effective if it gives rise to IgG's with higher affinity towards the allergen, in this case wt-bet v1, rather than to higher levels of IgGs.

Further, even though many animals can be used as models of human allergy, there are no validated animal models of allergy in which the effect of SIT can be assessed by symptomatic scoring of the animals. Hence, there is a need for finding surrogate endpoints for predictions of clinical performance in humans of variations to the vaccination regime (i.e. variations in the composition used or in its administration), in order to choose the optimal regime. Measurement of IgG affinity of anti-target antibodies generated in animals represents such a sought-after surrogate endpoint.

Such an animal model system can be used as a method for testing, identifying and selecting an immunotherapeutic preparation having improved properties as a vaccine against a target antigen, said method comprising raising antibodies against a group of candidate preparations and selecting the preparation which results in antibodies having higher affinity against the target antigen than does antibodies raised against at least one other preparation from the group.

The model system can also be used as a method for selecting an improved immunotherapeutic administration regime for a vaccine against a target antigen, said method comprising raising antibodies against the vaccine through a group of candidate administration regimes selecting the administration regime which results in antibodies having the higher affinity against the target antigen than does antibodies raised by at least one other administration regime from the group.

Preferentially, the improved preparation of vaccination antigen gives rise to antibodies of higher affinity than does a similar preparation with the target antigen. Still more preferred, the improved preparation or administration regime gives rise to antibodies of the highest affinity among the preparations and administration regimes tested.

The present invention relating to a method for identifying and selecting improved immunotherapeutic preparations to be included in a formulated vaccine against a target antigen, by measuring or determining the affinity of immunoglobulins towards the target antigen can be used in order to optimize by protein engineering the specific vaccination antigens to be included in the preparations to be compared. It will thus be possible according to the invention to compare the normally occurring target antigen with antigens in which changes to the peptide backbone has been introduced, thus evaluating if the changes have rendered the antigen more effective as a vaccination antigen. The preparations to be tested may also be tested for effects of different administration forms or addition of different adjuvants or other ingredients.

In one embodiment the vaccination antigens can be constructed by modifying a polypeptide (e.g. the target antigen) at specific amino acid positions identified by epitope mapping.

Thus the method of the invention includes identifying relevant positions for modification in the target antigen by epitope mapping, modifying the target antigen at relevant positions to produce variants, and including the variants in separate candidate preparations

### Epitope Mapping.

Vaccination antigen polypeptides may be epitope mapped by a number of methods, including those disclosed in detail in WO00/26230 and WO01/83559 and explained in the following. In brief, these methods comprises a database of epitope patterns (determined from an input of peptide sequences, known to bind specifically to anti-protein antibodies) and an algorithm to analyse 3-D structure of a given protein against the epitope pattern database. This will determine the possible epitopes on that protein, and the preference of each amino acid in the protein sequence to be part of epitopes.

### Identifying Antibody-Binding Peptides:

Antibody-binding peptides can be identified by many different ways. One is to synthesize a number of peptides of known sequence, and test for their ability to bind antibodies of interest, e.g., in ELISA or other immunochemical assays. Such data are available in great abundance in the literature.

A particularly effective way, is to prepare a library of many different random peptide sequences and select experimentally only the ones that bind antibodies well and specific (i.e., can be out-competed by the protein towards which the antibodies were raised). Phage display techniques are well suited for this way of finding antibody binding peptides:

In a phage display system, a sequence encoding a desired amino acid sequence is incorporated into a phage gene coding for a protein displayed on the surface of the phage. Thus, the phage will make and display the hybrid protein on its surface, where it can interact with specific target agents. Given that each phage contains codons for one specific sequence of a determined length, an average phage display library can express 10⁸-10¹² different random sequences. If the displayed sequence resembles an epitope, the phage can be selected by an epitope-specific antibody. Thus, it is possible to select specific phages from the bulk of a large number of phages, each expressing their one hybrid protein.

It is important that the amino acid sequence of the (oligo)peptides presented by the phage display system have a sufficient length to present a significant part of an epitope to be identified. The oligopeptides may have from 5 to 25 amino acids, preferably at least 8 amino acids, such as 9 amino acids.

The antibodies used for reacting with the oligopeptides can be polyclonal or monoclonal. In particular, they may be IgE antibodies to ensure that the epitopes identified are IgE epitopes, i.e., epitopes inducing and binding IgE. The antibodies may also be monospecific, meaning they are isolated according to their specificity for a certain protein. Polyclonal antibodies are preferred for building up data on antibody-binding peptides to be used in the in silico mapping tool in order to obtain a broader knowledge about the epitopes of a polypeptide.

These reactive peptides, by virtue of their reactivity against antibodies, to some degree resemble the appearance of an epitope on a full polypeptide.

Identifying Epitope Patterns from Reactive Peptides

The reactive (oligo)peptides identified e.g. by phage display are compared and aligned in order to identify common epitope patterns, which then can be used for identification of antibody binding epitopes on a 3-dimensional polypeptide.

In the alignment conservative alternatives to an amino acid such as aspartate and glutamate, lysine and arginine, serine and threonine are considered as one or equal.

Thus, the alignment results in a number of patterns, which depend on the chosen number of residues of the peptides. Using for example a 7-mer peptide, the pattern may have the form:
XX**XXX,
   where “*” in this notation indicates a non-consensus moiety which may be any amino acid or group of amino acids or no amino acid, while X is one of the following 13 residue types: AG, C, DE, FY, H, IL, KR, M, NQ, P, ST, V, and W, where the pairs AG, DE, FY, IL, KR, NQ, ST are conservative alternatives and considered equal. Accordingly, 3 peptides such as
A K S N N K R
A K S M N K R
A K T P N K K
   would create a pattern of [AG] [KR] [ST] * [NQ] [KR] [KR], where the residues AG KR ST and NQ KR KR are consensus residues shared by all 3 peptides and thus the epitope pattern would be AG KR ST* NQ KR KR. The patterns are chosen to describe a complete set of reactive (oligo)peptides (obtained e.g., by a phage display and antibody reaction) by the fewest possible patterns.

The epitope patterns may be determined directly from the reactive peptides; if for example a library of 7-mer reactive peptides is made, one can use each different reactive 7 mer peptide, taking conservative alternatives into account, as an epitope pattern in the epitope mapping approach as described below.

It is also possible to reduce the number of epitope patterns to be examined in the epitope mapping by removing redundant patterns and/or by employing experimental designs as known in the art (See example 1).

Within the identified epitope patterns some amino acids are conservative, called anchor amino acids. The anchor amino acids recur in all or a majority of the reactive peptides.

### Epitope Mapping Algorithm

When epitope patterns have been identified they are subsequently compared to the three-dimensional coordinates of the amino acid sequence of the polypeptide of interest, in order to identify combinations of residues on the polypeptide surface corresponding to the consensus sequence(s) or epitope pattern(s). In this way, amino acids residues, which are important for antibody binding, can be identified.

Once one or more epitope patterns have been identified, any polypeptide for which a three-dimensional structure is known may be analysed for epitopes matching the epitope patterns. Finding an epitope on a polypeptide is achieved by searching the surface of the polypeptide in the following way:

(1) For all amino acids in the polypeptide it is examined if (a) the amino acid type match the first amino acid of an epitope pattern and (b) the surface accessibility greater than or equal to a chosen threshold allowing the amino acid to be immunological interactive. Those amino acid satisfying 1(a) and 1(b) are selected.
(2) For all amino acids within a selected distance (e.g. 10 Angstroms) of the amino acids selected in step 1 it is examined if (a) the amino acid type matches the second amino acid of the pattern and (b) the surface accessibility greater than or equal to a chosen threshold allowing the amino add to be immunological interactive. Those amino acid satisfying 2(a) and 2(b) are selected
(3) For all amino acids within a selected distance (e.g., 10 Angstroms) of the amino acids selected in step 2 it is examined if (a) the amino add type matches the third amino acid of the pattern and (b) the surface accessibility greater than or equal to a chosen threshold allowing the amino acid to be immunological interactive. Those amino acid satisfying 3(a) and 3(b) are selected.

This procedure (step 3) is repeated for all amino acids in the epitope pattern consensus sequence. The coordinates of its C-alpha atom define the spatial positioning of an amino acid. The surface solvent accessibility threshold is given in percent of an average for the particular residue type (see example 2).

If matching amino acids for all amino acids in the epitope pattern can be found in the structure of the polypeptide it is a very strong indication that an epitope has been found. However it is also checked that the size of the epitope is satisfactory, i.e., the distance between any two residues is below a given threshold, usually 25 Å.

The epitopes found may be ranked and weighted according to their total accessible surface area, in order to improve further the predictability of the tool.

Finally, when all possible epitopes have been mapped for the protein of interest, one can provide a score for each amino acid of the protein by adding up the number of times it appears in an epitope pattern. This score will be an indication of the likelihood that modification (substitution, insertion, deletion, glycosylation or chemical conjugation) of that amino acid will, result in a variant with a lower antigenicity. All amino acids of the protein can then be ranked according to this score and those with highest scores can be selected for mutagenesis.

The epitope mapping tool can be adjusted, such that only a subset of the known reactive peptides are included as data set for building epitope patterns, and thus for conducting epitope mapping. For instance, one may choose only to include peptides reactive to IgE antibodies (rather than to IgG or other antibodies), or one may include only peptides reactive to human antibodies etc. One may choose to involve only peptides reactive against the target protein in order to get a more specific response, however, in general, peptides reactive to antibodies that in turn were raised against any protein are included.

If no three-dimensional structure coordinates are available for the protein of interest, one can map the epitope patterns directly to the primary sequence of the protein of interest.

From all the above information, it is obvious, that the epitopes are conveniently determined using this epitope mapping tool.

Further, the in silico epitope mapping tool can be used to predict if mutating one amino acid residue will result in that the new variant overall will have fewer epitopes. Thus, some or all 19 possible substitutions can be tested in a given position, the epitope mapping procedure repeated for a model structure of each of these proposed variants, and the best variant(s) can be constructed by mutation and tested experimentally.

In one embodiment of the invention at least two preparations are tested by the method of the present invention.

The method according to the invention in this embodiment comprises the following steps:

a) providing two or more vaccine preparations,
b) immunizing animals or groups of animals with one preparation each,
c) measuring target antigen affinity towards serum immunoglobulin isolated from the animals,
d) selecting one or more preparations resulting in higher immunoglobulin affinity towards the target antigen than at least one other preparation.
Preferably the preparation producing the highest affinity immunoglobulin is selected.

The two or more preparations can be different variants of the vaccination antigen. It can also be one antigen formulated with different adjuvants, or it can be one antigen subjected to various physical or chemical modification steps e.g. to create allergoids or desired fragmentation.

Alternatively, the composition can be the same, but two different administration regimes are tested (subcutaneous vs. intramuscular injections etc.).

In that case the present invention in another embodiment relates to a method for selecting one or more improved immunotherapeutic administration regimes for a vaccine against a target antigen, said method comprising raising antibodies against the vaccine through a group of candidate administration regimes and selecting one or more administration regimes having higher affinity against the target antigen than antibodies raised against at least one other administration regime from the group.

In a further embodiment the above method comprises the steps:

a) providing one or more vaccine preparations,
b) immunizing animals or groups of animals, each by different administration regime,
c) measuring target antigen affinity towards serum immunoglobulin isolated from the animals,
d) selecting one or more administration regimes or combinations of preparation and administration regime resulting in antibodies, preferably immunoglobulins, having higher affinity against the target antigen than antibodies raised against at least one of the tested administration regimes or regimes in combination with preparations. Preferably, the highest affinity regimes or regime preparation combination is selected.

Specifically, the target antigen may be an allergen (in the case of vaccination against an allergic disease). Other options can be, but is not limited to, antigens from pathogenic vira, microorganisms, or parasites, cancer-derived molecules for therapeutic vaccination against cancer or even various hybrid molecules, e.g. human proteins coupled to a non-human T-cell epitope, such as pharmaccines (www.pharmexa.com).

Examples of these include, but are not limited to vaccines against: choleara, Epstein-Barr virus; E. coli; genital herpes, Helicobacter pylori, hepatitis B, hepatitis C, influenza virus, Lyme disease, malaria, corona virus, MMR-varicella, Streptococcus, Staphylococcus, Rous Sarcoma Virus, HIV, Herpes 2, Human Papilloma Virus, and rotavirus.

Examples of cancer vaccines are those against the following target antigens: HLA-B7 (Vical, San Diego, Calif.); G17 hormone (Aphton, Miami, Fla.); Carcioembryonic antigen peptide-1; GMK ganglioside antigen (Bristol-Myers Squibb, Princeton, N.J.); G nRH hormone (Aphton, Miami, Fla.); Her2 (Corixa, Seattle, Wash.); Human Papilloma Virus HPV 16, E and E7 peptides; K-ras proteinMART-1; MUC-1 peptide (Corixa, Seattle, Wash.); p53; RAS; proteinase 3 peptide; antigens from fowlpox virus (Therion Biologics, Cambridge, Mass.).

In the case of an allergy vaccine, the following target allergens, among others, are of interest:

### Environmental Allergens

The environmental allergens that are of interest for epitope mapping include allergens from trees, grasses, herbs, house dust mites, cockroaches, mammals, venoms, fungi, dandruff, food items, and other plants.

Plant pollen allergens include but are not limited to those of the orders of Fagales, Oleales, Pinales, Poales, Asterales, and Urticales; including those from Betula, Alnus, Corylus, Carpinus, Olea, Phleum, Lolium, Poa, Cynodon, Secale, Dactylis, Ambrosia and Artemisia. Examples of specific allergens are Aln g1, Cor a1, Car b1, Cry j1, Amb a1 and a2, Art v1, Par j1, Ole e1, Ave e1, cyn d1, dac g1, Fes p1, Hol l1, Lol p1, Lol p5, Pas n1, Phl p1, and p5, poa p1, p2, and p5, Sec c1, and c5 and Sor h1 and Bet v1 (WO 99/47680).

From fungi, the allergens include but are not limited to those from Cladosporium, Aspergillus, and Alternaria, such as Alt a1 and Cla h1.

Mite allergens include but are not limited to those from Dermatophagoides farinae and Dermatophagoides pteronys., such as Der f1 and f2, and Der p1 and p2 as well as Lep d1 and d2.

From mammals, relevant environmental allergens include but are not limited to those from cat, dog, and horse as well as from dandruff from the hair of those animals, such as Fel d1; Can f1; Equ c1; Equ c2; Equ c3.

Venum allergens include but are not limited to those from bee, wasp, and fire ants, such as as well as Apis m1 and m2, Ves g1, g2 and g5, Ves v1, v2, and v5 and Te Pol and Sol i1, i2, i3, and i4 allergens. Well-known examples of these are PLA2 and hyaluronidase from bee venom.

Food allergens include but are not limited to those from milk (lactoglobulin), egg (ovalbumin), peanuts, hazelnuts, wheat (alfa-amylase inhibitor), and cherry.

### Commercial Allergens:

Commercial allergens that are of interest for epitope mapping are synthetic or naturally occurring industrially produced peptide, polypeptides and proteins.

One class of commercial allergens are pharmaceutical polypeptides. The term “pharmaceutical polypeptides” is defined as polypeptides, including peptides, such as peptide hormones, proteins and/or enzymes, being physiologically active when introduced into the circulatory system of the body of humans and/or animals. Pharmaceutical polypeptides are potentially immunogenic as they are introduced into the circulatory system. Examples of “pharmaceutical polypeptides” contemplated according to the invention include insulin, ACTH, glucagon, somatostatin, somatotropin, thymosin, parathyroid hormone, pigmentary hormones, somatomedin, erythropoietin, luteinizing hormone, chorionic go-nadotropin, hypothalmic releasing factors, antidiuretic hormones, thyroid stimulating hormone, relaxin, interferon, thrombopoietin (TPO) and prolactin.

Another class of commercial allergens are antimicrobial peptides (AMP's). The AMP is generally a relatively short peptide, consisting of less than 100 amino acid residues, typically 20-80 residues. The antimicrobial peptide has bactericidal and/or fungicidal effect, and it may also have antiviral or antitumour effects. It generally has low cytotoxicity against normal mammalian cells. The antimicrobial peptide is generally highly cationic and hy-drophobic. It typically contains several arginine and lysine residues, and it may not contain a single glutamate or aspa-ratate. It usually contains a large proportion of hydrophobic residues. The peptide generally has an amphiphilic structure, with one surface being highly positive and the other hydropho-bic.

A still further class of commercial allergens are enzymes which are catalytic polypeptides and/or proteins. The enzyme classification employed in the present specification is in accordance with Recommendations (1992) of the Nomenclature Committee of the International Union of Biochemistry and Molecular Biology, Academic Press, Inc., 1992.

Accordingly the types of enzymes which may appropriately be incorporated in granules of the invention include oxidoreductases (EC 1.-.-.-), transferases (EC 2.-.-.-), hydrolases (EC 3.-.-.-), lyases (EC 4.-.-.-), isomerases (EC 5.-.-.-) and ligases (EC 6.-.-.-).

Preferred oxidoreductases in the context of the invention are peroxidases (EC 1.11.1), laccases (EC 1.10.3.2) and glucose oxidases (EC 1.1.3.4)]. An Example of a commercially available oxidoreductase (EC 1.-.-.-) is GLUZYME® (enzyme available from Novozymes A/S).

Preferred transferases are transferases in any of the following sub-classes:

a) Transferases transferring one-carbon groups (EC 2.1);
b) transferases transferring aldehyde or ketone residues (EC 2.2); acyltransferases (EC 2.3);
c) glycosyltransferases (EC 2.4);
d) transferases transferring alkyl or aryl groups, other that methyl groups (EC 2.5); and
e) transferases transferring nitrogeneous groups (EC 2.6).

A most preferred type of transferase in the context of the invention is a transglutaminase (protein-glutamine gamma-glutamyltransferase; EC 2.3.2.13).

Further examples of suitable transglutaminases are described in WO 96/06931 (Novo Nordisk A/S).

Preferred hydrolases in the context of the invention are: Carboxylic ester hydrolases (EC 3.1.1.-) such as lipases (EC 3.1.1.3); phytases (EC 3.1.3.-), e.g. 3-phytases (EC 3.1.3.8) and 6-phytases (EC 3.1.3.26); glycosidases (EC 3.2, which fall within a group denoted herein as “carbohydrases”), such as alpha-amylases (EC 3.2.1.1); peptidases (EC 3.4, also known as proteases); and other carbonyl hydrolases].

In the present context, the term “carbohydrase” is used to denote not only enzymes capable of breaking down carbohydrate chains (e.g. starches or cellulose) of especially five- and six-membered ring structures (i.e. glycosidases, EC 3.2), but also enzymes capable of isomerizing carbohydrates, e.g. six-membered ring structures such as D-glucose to five-membered ring structures such as D-fructose.

Carbohydrases of relevance include the following (EC numbers in parentheses): alpha-amylases (EC 3.2.1.1), beta-amylases (EC 3.2.1.2), glucan 1,4-alpha-glucosidases (EC 3.2.1.3), endo-1,4-beta-glucanase (cellulases, EC 3.2.1.4), endo-1,3(4)-beta-glucanases (EC 3.2.1.6), endo-1,4-beta-xylanases (EC 3.2.1.8), dextranases (EC 3.2.1.11), chitinases (EC 3.2.1.14), poly-galacturonases (EC 3.2.1.15), lysozymes (EC 3.2.1.17), alpha-glucosidases (EC 3.2.1.21), alpha-galactosidases (EC 3.2.1.22), beta-galactosidases (EC 3.2.1.23), amylo-1,6-glucosidases (EC 3.2.1.33), xylan 1,4-beta-xylosidases (EC 3.2.1.37), glucan endo-1,3-beta-D-glucosidases (EC 3.2.1.39), alpha-dextrin endo-1,6-alpha-glucosidases (EC 3.2.1.41), sucrose alpha-glucosidases (EC 3.2.1.48), glucan endo-1,3-alpha-glucosidases (EC 3.2.1.59), glucan 1,4-beta-glucosidases (EC 3.2.1.74), glucan endo-1,6-beta-glucosidases (EC 3.2.1.75), arabinan endo-1,5-alpha-L-arabinosidases (EC 3.2.1.99), lactases (EC 3.2.1.108), chitosanases (EC 3.2.1.132) and xy-lose isomerases (EC 5.3.1.5).

Suitable proteases include those of animal, vegetable or microbial origin. Microbial origin is preferred. Chemically modified or protein engineered mutants are included. The protease may be a serine protease or a metallo-protease, preferably an alkaline microbial protease or a trypsin-like protease. Examples of alkaline proteases are subtilisins, especially those de-rived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946

Examples of commercially available proteases (peptidases) include KANNASE™, EVERLASE™, OVOZYME™, SAVOZYME™, ESPERASE™, ALCALASE™, NEUTRASE™, DURALASE™, DURAZYM™, SAVINASE™, PRIMASE™, PYRASE™, Pancreatic Trypsin NOVO (PTN), BIO-FEED™ PRO and CLEAR-LENS™ PRO (all available from Novozymes A/S).

Other commercially available proteases include MAXATASE™, MAXACAL™, MAXAPEM™, OPTICLEAN™, PROSPERASE™, PURAFECT™, PURAFECT OXP™, FN2™, FN3™. FN4™ and BLAP (available from Genencor International Inc., DSM or Henkel).

Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutz-eri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079 and WO 97/07202.

Examples of commercially available lipases include LIPEX, LIPOPRIME™, LIPOLASE™, LIPOLASE™ Ultra, LIPOZYME™, PALATASE™, NOVOZYM™ 435 and LECITASE™ (all available from Novozymes A/S).

Other commercially available lipases include LUMAFAST™ (Pseudomonas mendocina lipase from Genencor International Inc.); LIPOMAX™ (Ps. pseudoalcaligenes lipase from DSM/Genencor Int. Inc.; and Bacillus sp. lipase from Genencor enzymes. Further lipases are available from other suppliers.

Examples of commercially available carbohydrases Include ALPHA-GAL™, BIO-FEED™ Alpha, BIO-FEED™ Beta, BIO-FEED™ Plus, BIO-FEED™ Plus, NOVOZYME™ 188, CELLUCLAST™, CELLUSOFT™, CEREMYL™, CITROZYM™, DENIMAX™, DEZYME™, DEXTROZYME“, FINIZYM”, FUNGAMYL™, GAMANASE™, GLUCANEX™, LAC-TOZYM™, MALTOGENASE™, PENTOPAN™, PECTINEX™, PROMOZYMEM™, PULPZYME™, NOVAMYL™, TERMAMYL™, AMG™ (Amyloglucosidase Novo), MALTOGENASE™, SWEETZYME™ and AQUAZYM™ (all available from Novozymes A/S). Further carbohydrases are available from other suppliers.

Suitable amylases (alpha and/or beta) include those of bacterial or fungal origin. Chemically modified or protein engineered mutants are included. Amylases include, for example, alpha-amylases obtained from Bacillus, e.g. a special strain of B. licheniformis, described in more de-tail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are NATALASE™, STAINZYME™, DURAMYL™, TERMAMYL™, TERMAMYL™ ULTRA, FUNGAMYL™ and BAN™ (Novozymes A/S), RAPI-DASE™PURASTAR™ and PURASTAR OXAM™ (from Genencor International Inc.). Suitable cellulases include those of bacterial or fungal origin. Chemically modi-fied or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Commercially available cellulases include CELLUZYME™, ENDOLASE™RENOZYME™ and CAREZYME™ (Novozymes A/S), CLAZINASE™, and PURADAX HA™ (Genencor Interna-tional Inc.), and KAC-500(B)™ (Kao Corporation). Oxidoreductases: Particular oxidoreductases in the context of the invention are peroxidases (EC 1.11.1), laccases (EC 1.10.3.2) and glucose oxidases (EC 1.1.3.4)]. An Example of a commercially available oxidoreductase (EC 1.-.-.-) is GLUZYME™ (enzyme available from Novozymes A/S). Further oxidoreductases are available from other suppliers.

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g. from C. cinereus, and variants thereof as those described in WO 93/24618, WO 95/10602, and WO 98/15257. Commercially available peroxidases include GUARDZYME™ (Novozymes A/S).

Any mannanase suitable for use in alkaline solutions can be used. Suitable mannanases include those of bacterial or fungal origin. Chemically or genetically modified mutants are included.

In a preferred embodiment the mannanase is derived from a strain of the genus Bacillus, especially Bacillus sp. I633 disclosed in positions 31-330 of SEQ ID NO:2 or in SEQ ID NO: 5 of WO 99/64619 or Bacillus agaradhaerens, for example from the type strain deposited DSM 8721. In a more preferred embodiment of the present invention the mannanase is derived from Alkalophilic bacillus. Suitable mannanases include MANNAWAY™ (Novozymes A/S).

Any pectate lyase suitable for use in alkaline solutions can be used. Suitable pectate lyases include those of bacterial or fungal origin. Chemically or genetically modified mutants are included.

In a preferred embodiment the pectate lyase is derived from a strain of the genus Bacil-lus, especially a strain of Bacillus substilis, especially Bacillus subtilis DSM14218 disclosed in SEQ ID NO:2 or a variant thereof disclosed in Example 6 of WO 02/092741. In a more pre-ferred embodiment of the present invention the pectate lyase is derived from Bacillus licheniformis.

Other commercial allergens are those from plants, such as latex (hevea brasiliensis).

In an optional step, it may be of interest to pre-select variants based on their affinity to IgG antibodies (e.g. anti-reference protein or anti-target antigen) in vitro as a simple first step to reduce the number of proposed variants that should be tested in animals. This can be done by measuring affinity towards such antibody samples that are deemed relevant for the targeted allergic disease.

### Measuring Affinity.

The affinity of antigen to antibodies from serum can be determined by surface plasmon resonance (SPR). In the SPR detection system one of the interactants is attached to a sensor surface, while the other flows in solution over the surface. An SPR detection system (such as the Biacore™ instrument) monitors biomolecular interaction by measuring the refractive index close to the surface of the sensor chip. As molecules bind to the sensor chip, the refractive index changes, and an SPR response is observed, which is proportional to the mass of material that has bound.

During sample injection, the concentration of free analyte [A] (a.g. antibody or antigen) is kept constant by continuous supply of new sample (C), and the concentration of complex on the surface [AB] (allergen-Ab) is given in resonance units (RU) by the response above baseline (R). The concentration of free ligand on the surface [B] (allergen) is not measured directly. However, the total ligand concentration can be expressed in resonance units as the maximum analyte binding capacity Rmax and the concentration of free ligand is then Rmax-R.

In a bimolecular binding event, the appropriate reactions are: A+B->AB, with the rate constant kon; and AB->A+B with rate constant koff. Thus the affinity constant Ka=[AB]/([A]*[B]).

In the biacore experiment, ratio of bound to free ligand at the surface [AB] is substituted by R, [B] is substituted by (Rmax-R), and [A] is substituted by C. Thus,

dR/dt=kon*C*(Rmax−R)−koff*R

Since the terms R and Rmax are both measured in R, it does not matter that the molar concentrations of ligand and complex on the surface are not known. The concentration, of analyte (C) must however be known in molar terms.

After the sample has passed over the surface, analyte that dissociates from the complex is carried away by the buffer flow, so that the free analyte concentration is zero. The rate of dissociation is then described by the equation:

dR/dt=−koff*R

This theoretical treatment applies to the ideal case of a homogeneous 1:1 interaction between ligand and analyte, with a fully efficient supply of analyte during the sample injection and a fully efficient removal of free analyte in the dissociation phase.

In the case of antigen-antibody interaction, the binding is bivalent, and a polyclonal antibody sample contains many molecular entities (the different clones of the polyclonal antibody), hence the SPR analysis gives only an apparent binding constants when analysed according to above method. This apparent Kon, koff, and Ka are used throughout this patent text.

Other measures of binding affinity are well known in the art. As long as the protein concentrations of both binding partners are known, one can use several methods. For most methods, this requires purification and quantification or immobilization of serum antibodies.

The methods include general binding assays analysed by Scatchard or Hill plots (Dahlquist, Methods of Enzymol, vol. 48, pp. 270-299, 1978) or displacement curves (Horovits et al, PNAS; vol. 84, pp. 6654-6658, 1987) or fitted isotherms (Brodersen et al, Eur. J. Biochem, vol. 169 ppp. 487.495, 1987) or differential scanning calorimetry (Brandts and Lin, Biochemistry, vol. 29, pp. 6927-40, 1990). These disclosures are incorporated by reference.

An example of this using iodine-125 labelled allergen (bet v 1) is provided by Svenson et al, Mol. Immunology, vol. 39, pp 603-12, 2003.

Further the deselection step can be done by ELISA based methods (see below at ‘Direct ELISA’), which do not necessarily give a binding constant as a direct result, but which still provide a relative ranking of the various antigens for their ability to bind relevant antibody.

### Competitive ELISA:

A competitive ELISA can give a direct comparison of two potential vaccination antigens with respect to their ability to bind relevant antibodies (e.g. human anti-target serum, IgG, IgG1, or IgG4) in order to preselect the best candidates to go into animal or clinical studies. It is carried out like direct ELISA (see below), with two exceptions: the immunoplates are coated with a fixed concentration of a reference polypeptide (which can for example be the target antigen or one of the vaccination antigens), and the diluted patient or animal serum is pre-incubated with a dose range of the vaccination antigen candidates or reference proteins. If the antibodies bind to the polypeptide in solution, it will reduce binding to the reference protein bound to the plate, thus reducing the OD450. Candidate vaccination antigens are selected, which demonstrate increased binding interactions (including increased affinity) for the anti-target antigen antibody.

### Animal Models of Allergy and SIT/Immunise Experimental Animals With Each of the Compositions:

The in vivo immunogenic properties of the polypeptide variant of the invention may suitably be measured in an animal test, wherein test animals are exposed to a vaccination allergen polypeptide and the responses are measured and compared to those of the target allergen or other appropriate references. The immune response measurements may include comparing reactivity of serum IgG, IgE or T-cells from a test animal with target polypeptide and the polypeptide variant. Animal immunization can be conducted in at least two distinct manners: on naïve animals and on pre-sensitized animals (to better simulate the vaccine situation). In the context of this invention affinity of immunoglobulins towards the target antigen is tested.

In a particular embodiment the affinity of animal IgG and/or IgG1 and/or IgG4 following administration of the variant molecule is tested.

In the method according to the invention the test animals can either be naïve animals or pre-sensitized animals.

A number of model systems are based on the use of naïve animals:

In a particular embodiment the in vivo verification comprises exposing a mouse to a parent target allergen by the intranasal route. Useful in vivo animal models include the mouse intranasal test (MINT) model (Robinson et al., Fund. Appl. Toxicol. 34, pp. 15-24, 1996).

In a further particular embodiment the in vivo verification comprises exposing a test animal to a polypeptide variant by the intratracheal route. Useful in vivo animal models include the guinea pig intratracheal (GPIT) model (Ritz, et al. Fund. Appl. Toxicol., 21, pp. 31-37, 1993) and the rat intratracheal (rat-IT) model (WO 96/17929, Novo Nordisk).

In a further particular embodiment, the in vivo verification comprises exposing a test animal subcutaneously to the target allergen and the vaccination allergen variant. A suitable model is the mouse subcutaneous (mouse-SC) model (WO 98/30682, Novo Nordisk).

In a further particular embodiment, the method comprise exposing the test animal intraperitoneally. ALK-Abelló disclose (WO02/40676) a method to assess the ability of allergen variants (of the birch pollen allergen bet v 1) to induce IgG antibodies upon immunization of mice: BALB/C mice were immunized intraperitoneally with the relevant allergy variant or controls, four times at dose intervals of 14 days. The proteins were conjugated to 1,25 mg/ml alhydrogel (AlOH gel, 1,3%, pH8-8.4, Superfos Biosector). The mice were immunized with either 1 or 10 ug protein/dose. Blood samples were drawn at day 0, 14, 21, 35, 49 and 63, and analysed by direct ELISA using rBet v 1 coated microtiterplates and biotinylated rabbit anti mouse IgG antibodies as detecting antibodies.

In yet a further embodiment, the method comprise using transgenic mice capable of facilitating production of donor-specific immunity as test animals. Such mice are disclosed by Genencor International (WO 01/15521)

Also, a number of studies have assessed the effect of allergy vaccination compositions in animal models, in which the animals were sensitized to the relevant allergen prior to exposure to the vaccination composition:

Mice: Li et al. (J. Allergy Clin. Immunol., vol. 112, pp 159-167, 2003) disclose a mice-based system to assess efficacy of allergy vaccines. The mice are sensitized intra-gastrically with a food allergen, and the treatment is introduced as an intra-rectum injection. In a separate allergy vaccination system, Hardy et al. (AM J. Respir. Crit. Care Med, vol 167, pp. 1393-1399, 2003) show that mice can be sensitized by intraperitonal injection, and that allergy vaccine compounds can be administered intratracheally with the animals anaestethized. Sudowe et al., (Gene Therapy, vol. 9, 147-156, 2002) show that intraperitoneal injection in mice could be made to produce either TH1 or TH2 responses.

Rats: Wheeler et al., (Int. Arch. Allergy Immunol, vol. 126, pp. 135-139, 2001) disclose a rat allergy model in which rats are injected subcutaneously along with adjuvant. These ‘allergic’ rats can then be made to conduct an allergy-vaccine like response, when subjected to subsequent injections with trial vaccine compositions.

Guinea Pigs: Nakamoto et al., (Clin Exp. Allergy, vol. 27, pp 1103-1108 1997) demonstrate the use of guinea pigs as model system for SIT. Guinea pigs were injected intraperitoneally and boosted twice, and then they were exposed to the vaccine compound to register decreases in ‘allergenicity’ by measuring antibody titers as a function of the compound, formulation, or mode of application.

Isolating Sub-Fractions of Serum Ig (e.g. Specific IgG/IgG1/IgG4)

In one embodiment of the invention a sub-fraction of serum immunoglobulin is isolated before step (c) above.

The sub-fractions comprise in one embodiment specific IgG, IgG1 and/or IgG4.

There are a wide variety of methods used to purify antibodies. Methods conventionally used include precipitation and column chromatography. Protein A and Protein G coupled columns or beads are used for purifying human and mouse IgG1 and IgG4 from serum, ascites or cell culture media. The immobilised antibodies are typically eluted by lowering the pH of the buffer.

One useful approach for separating antibody-subtypes (IgG1, IgG4) or for purifying IgE (which does not bind to Protein A or G) from serum in a single step, is to purify them over an anti-IgX antibody column, i.e. a column with immobilized antibodies specific for one of the immunoglobulin subclasses from the rellevant species e.g. anti-mouse-IgG1. The column or beads are washed and the desired antibodies are eluted by treatment with a low pH buffer and then a high pH buffer. Any antibodies held by bonds that are broken under these conditions will be eluted and are available for further tests. (Antibodies. A laboratory manual (Eds Harlow E, Lane D-Cold Spring Harbour-1988)

Measuring Apparent Affinity of the Target Antigen Towards Serum Ig from the Immunized Animal.

The apparent affinity of the antigen interaction with antibody raised in serum of test animal can be determined by any of the methods described above (‘Measuring Affinity’)

From the animal exposure studies, it may also be of interest to measure the serum concentration of IgG and/or IgE: For allergy vaccination uses, vaccination antigens that lead to formation of IgG1 and especially IgG4, of higher apparent affinity for the target antigen, and also to reduced formation of anti-target antigen IgE are of special interest.

### Direct ELISA for Measurement of the Concentration of Specific IgG1 in Mouse Serum

The relative concentrations of specific IgG1 antibodies in serum samples from mice are measured by a three layer sandwich ELISA according to the following procedure:
1) The ELISA-plate (Nunc Maxisorp) is coated with 100 microliter/well of vaccination antigen polypeptide variant diluted in PBS to 10 microgram/ml. Incubated over night at 4° C.
2) The wells are emptied and blocked with 200 microliter/well 2% skim milk in 0.15 M PBS buffer pH 7.5 for 1 hour at 4° C. The plates are washed 3 times with 0.15 M PBS buffer with 0.05% Tween20.
3) The plates are incubated with dilutions of mouse sera (100 microliter/well), starting from an 20-fold dilution and 3-fold dilutions hereof in 0.15 M PBS buffer with 0.5% skim milk and 0.05% Tween20. Appropriate dilutions of positive and negative control serum samples plus buffer controls are included. Incubated for 1 hour at room temperature. Gently shaken. The plates were washed as before.
4) 100 microliter/well biotinylated Rat-anti-mouse IgG₁, (Serotec, Cat. No.: MCA 336B), diluted 2000x in 0.15 M PBS buffer with 0.5% skim milk and 0.05% Tween20 is added to the plates. The plates are incubated for 1 hour at 4° C. The plates are washed as before.
5) 100 microliter/well Horseradish Peroxidase-conjugated Streptavidin (Kierkegaard & Perry, Cat. No.: 14-30-00), diluted 1000x in 0.15 M PBS buffer with 0.15% skim milk and 0.05% Tween20. Incubated for 1 hour at 4° C. The plates are washed as before.
6) 100 μl/well TMB (Plus (Ready-to-go substrate; Kem-En-Tec, Cat. No.: 4390A) is added, and the reaction is allowed to run for 10 min.
7) The reaction is stopped by adding 100 microliter/well 1M H₂SO₄
8) The plates are read at 450 nm with 620 nm as reference.
   Specific IgG4 and IgE are detected similarly, but by using the relevant detection antibody in step 4 (e.g. Rat-anti-mouse IgG4, rat anti-mouse IgE, or rabbit anti-mouse IgE (DAKOcytomation, Glostrup, Denmark). Also, variations of this method can be implemented for other animal species than mice, in which case suitable detection antibodies must be chosen.
   Selecting Compositions that Gives Rise to High Ig Affinity Towards the Target Antigen.

In a final step, the test results for at least two test variants are compared and the one or ones with best IgG1 and/or IgG4 affinity for target antigen are chosen for further analysis.

Furthermore, the selection criteria can include a measure of the vaccination antigen's ability to functionally bind human anti-target antigen IgE. For allergy vaccines, the patient to be vaccinated will have circulating IgE, and it is important for the vaccination antigen to have as small a propensity as possible to elicit immediate or late phase allergic reactions upon vaccination. Thus, the vaccination antigen candidates can be tested in a histamine release assay with patient anti-target antigen serum (containing relevant IgE) (see e.g., Nolte et al., Allergy, vol. 42, pp. 366-373,1987). The vaccination antigens that give the lowest response in this assay are preferred as vaccine candidates.

### Basophil Histamine Release:

The basophil containing cell fraction is isolated from whole blood from donors allergic to the target allergen, by centrifugation. The cells are then incubated with a dose range of a candidate vaccination allergen. IgE binding will crosslink IgE on the surface of the basophile granulocytes, thereby releasing histamine into the surroundings. Liberated histamine can then be measured by, e.g., fluorometric methods (see e.g., Nolte et al., Allergy, vol. 42, pp. 366-373,1987).

### Preparations of Variant Genes, Cells Expressing Variant Protein, and Purification of Variant Protein

### Nucleotide Sequences:

The present invention also encompasses a nucleotide sequence encoding a polypeptide variant of the invention. As described, a description of standard mutation of nucleotide sequences to encode polypeptide variants by nucleotide substitution can be found in e.g., Ford et al., 1991, Protein Expression and Purification 2, p. 95-107. Other standard methods, such as site-directed mutagenesis is described in e.g., Sambrook et al. (1989), Molecular Cloning. A Laboratory Manual, Cold Spring Harbor, N.Y.

A “nucleotide sequence” is a single- or double-stranded polymer of deoxyribonucleotide or ribonucleotide bases read from the 5' to the 3' end. Nucleotide sequences include RNA and DNA, and may be isolated from natural sources, synthesized in vitro, or prepared from a combination of natural and synthetic molecules.

The techniques used to isolate or clone a nucleotide sequence alias a nucleotide sequence encoding a polypeptide are known in the art and include isolation from genomic DNA, preparation from cDNA, or a combination thereof. The cloning of the nucleotide sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, A Guide to Methods and Application, Academic Press, New York. Other nucleotide amplification procedures such as ligase chain reaction (LCR), ligated activated transcription (LAT) and nucleic acid sequence-based amplification (NASBA) may be used. The nucleotide sequence may be cloned from a strain producing the polypeptide, or from another related organism and thus, for example, may be an allelic or species variant of the polypeptide encoding region of the nucleotide sequence.

The term “isolated” nucleotide sequence as used herein refers to a nucleotide sequence which is essentially free of other nucleotide sequences, e.g., at least about 20% pure, preferably at least about 40% pure, more preferably about 60% pure, even more preferably about 80% pure, most preferably about 90% pure, and even most preferably about 95% pure, as determined by agarose gel electorphoresis. For example, an isolated nucleotide sequence can be obtained by standard cloning procedures used in genetic engineering to relocate the nucleotide sequence from its natural location to a different site where it will be reproduced. The cloning procedures may involve excision and isolation of a desired nucleotide fragment comprising the nucleotide sequence encoding the polypeptide, insertion of the fragment into a vector molecule, and incorporation of the recombinant vector into a host cell where multiple copies or clones of the nucleotide sequence will be replicated. The nucleotide sequence may be of genomic, cDNA, RNA, semisynthetic, synthetic origin, or any combinations thereof. Such isolated molecules are those that are separated from their natural environment and include cDNA and genomic clones. Isolated DNA molecules of the present invention are free of other genes with which they are ordinarily associated, and may include naturally occurring 5' and 3' untranslated regions such as promoters and terminators. The identification of associated regions will be evident to one of ordinary skill in the art (see for example, Dynan and Tijan, Nature 316: 774-78, 1985).

### Nucleotide Construct:

As used herein the term “nucleotide construct” is intended to indicate any nucleotide molecule of cDNA, genomic DNA, synthetic DNA or RNA origin. The term “construct” is intended to indicate a nucleotide segment which may be single- or double-stranded, and which may be based on a complete or partial naturally occurring nucleotide sequence encoding a polypeptide of interest. The construct may optionally contain other nucleotide segments.

The DNA of interest may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., supra).

The nucleotide construct may also be prepared synthetically by established standard methods, e.g., the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesized, e.g., in an automatic DNA synthesizer, purified, annealed, ligated and cloned in suitable vectors.

Furthermore, the nucleotide construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleotide construct, in accordance with standard techniques.

The nucleotide construct may also be prepared by polymerase chain reaction using specific primers, for instance as described in U.S. Pat. No. 4,683,202 or Saiki et al., Science 239 (1988), 487-491.

The term nucleotide construct may be synonymous with the term expression cassette when the nucleotide construct contains all the control sequences required for expression of a coding sequence of the present invention.

The term “coding sequence” as defined herein is a sequence which is transcribed into mRNA and translated into a polypeptide of the present invention when placed under the control of the above mentioned control sequences. The boundaries of the coding sequence are generally determined by a translation start codon ATG at the 5'-terminus and a translation stop codon at the 3'-terminus. A coding sequence can include, but is not limited to, DNA, cDNA, and recombinant nucleotide sequences.

The term “control sequences” is defined herein to include all components which are necessary or advantageous for expression of the coding sequence of the nucleotide sequence. Each control sequence may be native or foreign to the nucleotide sequence encoding the polypeptide. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide. Transcriptional and translational control sequences are DNA regulatory sequences, such as promoters, enhancers, terminators, and the like, that provide for the expression of a coding sequence in a host cell. In eukaryotic cells, polyadenylation signals are control sequences.

The nucleotide constructs of the present invention may also comprise one or more nucleotide sequences which encode one or more factors that are advantageous in the expression of the polypeptide, e.g., an activator (e.g., a trans-acting factor), a chaperone, and a processing protease. Any factor that is functional in the host cell of choice may be used in the present invention. The nucleotides encoding one or more of these factors are not necessarily in tandem with the nucleotide sequence encoding the polypeptide.

### Propeptides:

The control sequence may also be a propeptide coding region, which codes for an amino acid sequence positioned at the amino terminus of a polypeptide. The resultant polypeptide is known as a proenzyme or propolypeptide (or a zymogen in some cases). A propolypeptide is generally inactive and can be converted to mature active polypeptide by catalytic or autocatalytic cleavage of the propeptide from the propolypeptide. The propeptide coding region may be obtained from the Bacillus subtilis alkaline protease gene (aprE), the Bacillus subtilis neutral protease gene (nprT), the Saccharomyces cerevisiae alpha-factor gene, or the Myceliophthora thermophilum laccase gene (WO 95/33836).

### Activators:

An activator is a protein which activates transcription of a nucleotide sequence encoding a polypeptide (Kudla et al., 1990, EMBO Journal 9:1355-1364; Jarai and Buxton, 1994, Current Genetics 26:2238-244; Verdier, 1990, Yeast 6:271-297). The nucleotide sequence encoding an activator may be obtained from the genes encoding Bacillus stearothermophilus NprA (nprA), Saccharomyces cerevisiae heme activator protein 1 (hap1), Saccharomyces cerevisiae galactose metabolizing protein 4 (gal4), and Aspergillus nidulans ammonia regulation protein (areA). For further examples, see Verdier, 1990, supra and MacKenzie et al., 1993, Journal of General Microbiology 139:2295-2307.

### Chaperones:

A chaperone is a protein which assists another polypeptide in folding properly (Hartl et al., 1994, TIBS19:20-25; Bergeron et al., 1994, TIBS19:124-128; Demolder et al., 1994, Journal of Biotechnology 32:179-189; Craig, 1993, Science 260:1902-1903; Gething and Sambrook, 1992, Nature 355:33-45; Puig and Gilbert, 1994, Journal of Biological Chemistry 269:7764-7771; Wang and Tsou, 1993, The FASEB Journal 7:1515-11157; Robinson et al., 1994, Bio/Technology 1:381-384). The nucleotide sequence encoding a chaperone may be obtained from the genes encoding Bacillus subtilis GroE proteins, Aspergillus oryzae protein disulphide isomerase, Saccharomyces cerevisiae calnexin, Saccharomyces cerevisiae BiP/GRP78, and Saccharomyces cerevisiae Hsp70. For further examples, see Gething and Sambrook, 1992, supra, and Hartl et al., 1994, supra.

### Processing Protease:

A processing protease is a protease that cleaves a propeptide to generate a mature biochemically active polypeptide (Enderlin and Ogrydziak, 1994, Yeast 10:67-79; Fuller et al., 1989, Proceedings of the National Academy of Sciences USA 86:1434-1438; Julius et al., 1984, Cell 37:1075-1089; Julius et al., 1983, Cell 32:839-852). The nucleotide sequence encoding a processing protease may be obtained from the genes encoding Aspergillus niger Kex2, Saccharomyces cerevisiae dipeptidylaminopeptidase, Saccharomyces cerevisiae Kex2, and Yarrowia lipolytica dibasic processing endoprotease (xpr6).

### Promoters:

The control sequence may be an appropriate promoter sequence, a nucleotide sequence which is recognized by a host cell for expression of the nucleotide sequence. The promoter sequence contains transcription and translation control sequences which mediate the expression of the polypeptide. The promoter may be any nucleotide sequence which shows transcriptional activity in the host cell of choice and may be obtained from genes encoding extracellular or intracellular polypeptides either homologous or heterologous to the host cell. The term “promoter” is used herein for its art-recognized meaning to denote a portion of a gene containing DNA sequences that provide for the binding of RNA polymerase and initiation of transcription. Promoter sequences are commonly, but not always, found in the 5' non-coding regions of genes.

Examples of suitable promoters for directing the transcription of the nucleotide constructs of the present invention, especially in a bacterial host cell, are the promoters obtained from the E. coli lac operon, the Streptomyces coelicolor agarase gene (dagA), the Bacillus subtilis levansucrase gene (sacB), the Bacillus subblis alkaline protease gene, the Bacillus licheniformis alpha-amylase gene (amyL), the Bacillus stearothermophilus maltogenic amylase gene (amyM), the Bacillus amyloliquefaciens alpha-amylase gene (amyQ), the Bacillus amyloliquefaciens BAN amylase gene, the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75:3727-3731), as well as the tac promoter (DeBoer et al., 1983, Proceedings of the National Academy of Sciences USA 80:21-25), or the Bacillus pumilus xylosidase gene, or by the phage Lambda PR or PL promoters or the E. coli lac, trp or tac promoters. Further promoters are described in “Useful proteins from recombinant bacteria” in Scientific American, 1980, 242:74-94; and in Sambrook et al., 1989, supra.

Examples of suitable promoters for directing the transcription of the nucleotide constructs of the present invention in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus a wamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral (-amylase and Aspergillus oryzae triose phosphate isomerase), and glaA promoters. Further suitable promoters for use in filamentous fungus host cells are the ADH3 promoter (McKnight et al., The EMBO J. 4 (1985), 2093-2099) or the tpiA promoter.

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4-c (Russell et al., Nature 304 (1983), 652-654) promoters.

Further useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8:423-488. In a mammalian host cell, useful promoters include viral promoters such as those from Simian Virus 40 (SV40), Rous sarcoma virus (RSV), adenovirus, and bovine papilloma virus (BPV).

Examples of suitable promoters for directing the transcription of the DNA encoding the polypeptide of the invention in mammalian cells are the SV40 promoter (Subramani et al., Mol. Cell. Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

### Terminators:

The control sequence may also be a suitable transcription terminator sequence, a sequence recognized by a host cell to terminate transcription. The terminator sequence is operably linked to the 3' terminus of the nucleotide sequence encoding the polypeptide. Any terminator which is functional in the host cell of choice may be used in the present invention.

Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. for fungal hosts) the TPI1 (Alber and Kawasaki, op. cit.) or ADH3 (McKnight et al., op. cit.) terminators.

Preferred terminators for yeast host cells are obtained from the genes encoding Saccharomyces cerevisiae enolase, Saccharomyces cerevisiae cytochrome C (CYC1), or Saccharomyces cerevisiae glyceraldehyde-3-phosphate dehydrogenase. Other useful terminators for yeast host cells are described by Romanos et al., 1992, supra.

### Polyadenylation Signals:

The control sequence may also be a polyadenylation sequence, a sequence which is operably linked to the 3' terminus of the nucleotide sequence and which, when transcribed, is recognized by the host cell as a signal to add polyadenosine residues to transcribed mRNA. Any polyadenylation sequence which is functional in the host cell of choice may be used in the present invention.

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase.

Useful polyadenylation sequences for yeast host cells are described by Guo and Sherman, 1995, Molecular Cellular Biology 15:5983-5990.

Polyadenylation sequences are well known in the art for mammalian host cells such as SV40 or the adenovirus 5 Elb region.

### Signal Sequences:

The control sequence may also be a signal peptide coding region, which codes for an amino acid sequence linked to the amino terminus of the polypeptide which can direct the expressed polypeptide into the cell's secretory pathway of the host cell. The 5' end of the coding sequence of the nucleotide sequence may inherently contain a signal peptide coding region naturally linked in translation reading frame with the segment of the coding region which encodes the secreted polypeptide. Alternatively, the 5' end of the coding sequence may contain a signal peptide coding region which is foreign to that portion of the coding sequence which encodes the secreted polypeptide. A foreign signal peptide coding region may be required where the coding sequence does not normally contain a signal peptide coding region. Alternatively, the foreign signal peptide coding region may simply replace the natural signal peptide coding region in order to obtain enhanced secretion relative to the natural signal peptide coding region normally associated with the coding sequence. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region capable of directing the expressed polypeptide into the secretory pathway of a host cell of choice may be used in the present invention.

A “secretory signal sequence” is a DNA sequence that encodes a polypeptide (a “secretory peptide” that, as a component of a larger polypeptide, directs the larger polypeptide through a secretory pathway of a cell in which it is synthesized. The larger polypeptide is commonly cleaved to remove the secretory peptide during transit through the secretory pathway.

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis PrsA gene. Further signal peptides are described by Simonen and Palva, 1993, Microbiological Reviews 57:109-137.

An effective signal peptide coding region for filamentous fungal host cells is the signal peptide coding region obtained from Aspergillus oryzae TAKA amylase gene, Aspergillus niger neutral amylase gene, the Rhizomucor miehei aspartic proteinase gene, the Humicola lanuginosa cellulase or lipase gene, or the Rhizomucor miehei lipase or protease gene, Aspergillus sp. amylase or glucoamylase, a gene encoding a Rhizomucor miehei lipase or protease. The signal peptide is preferably derived from a gene encoding A. oryzae TAKA amylase, A. niger neutral (-amylase, A. niger acid-stable amylase, or A. niger glucoamylase. Useful signal peptides for yeast host cells are obtained from the genes for Saccharomyces cerevisiae a-factor and Saccharomyces cerevisiae invertase. Other useful signal peptide coding regions are described by Romanos et al., 1992, supra.

For secretion from yeast cells, the secretory signal sequence may encode any signal peptide which ensures efficient direction of the expressed polypeptide into the secretory pathway of the cell. The signal peptide may be naturally occurring signal peptide, or a functional part thereof, or it may be a synthetic peptide. Suitable signal peptides have been found to be the a-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6,1990, pp. 127-137).

For efficient secretion in yeast, a sequence encoding a leader peptide may also be inserted downstream of the signal sequence and upstream of the DNA sequence encoding the polypeptide. The function of the leader peptide is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The leader peptide may be the yeast a-factor leader (the use of which is described in e.g., U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

### Other Regulator Sequences:

It may also be desirable to add regulatory sequences which allow the regulation of the expression of the polypeptide relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GALL system may be used. In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those which allow for gene amplification. In eukaryotic systems, these include the dihydrofolate reductase gene which is amplified in the presence of is methotrexate, and the metallothionein genes which are amplified with heavy metals. In these cases, the nucleotide sequence encoding the polypeptide would be placed in tandem with the regulatory sequence.

### Recombinant Expression Vector Comprising Nucleotide Construct:

The present invention also relates to a recombinant expression vector comprising a nucleotide sequence of the present invention, a promoter, and transcriptional and translational stop signals. The various nucleotide and control sequences described above may be joined together to produce a recombinant expression vector which may include one or more convenient restriction sites to allow for insertion or substitution of the nucleotide sequence encoding the polypeptide at such sites. Alternatively, the nucleotide sequence of the present invention may be expressed by inserting the nucleotide sequence or a nucleotide construct comprising the sequence into an appropriate vector for expression. In creating the expression vector, the coding sequence is located in the vector so that the coding sequence is operably linked with the appropriate control sequences for expression, and possibly secretion.

“Operably linked”, when referring to DNA segments, indicates that the segments are arranged so that they function in concert for their intended purposes, e.g., transcription initiates in the promoter and proceeds through the coding segment to the terminator.

An “Expression vector” is a DNA molecule, linear or circular, that comprises a segment encoding a polypeptide of interest operably linked to additional segments that provide for its transcription. Such additional segments may include promoter and terminator sequences, and optionally one or more origins of replication, one or more selectable markers, an enhancer, a polyadenylation signal, and the like. Expression vectors are generally derived from plasmid or viral DNA, or may contain elements of both.

The recombinant expression vector may be any vector (e.g., a plasmid or virus), which can be conveniently subjected to recombinant DNA procedures and can bring about the expression of the nucleotide sequence. The choice of the vector will typically depend on the compatibility of the vector with the host cell into which the vector is to be introduced. The vectors may be linear or closed circular plasmids. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol, tetracycline, neomycin, hygromycin or methotrexate resistance. A frequently used mammalian marker is the dihydrofolate reductase gene (DHFR). Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5'-phosphate decarboxylase), sC (sulfate adenyltransferase), trpC (anthranilate synthase), and glufosinate resistance markers, as well as equivalents from other species. Preferred for use in an Aspergillus cell are the amdS and pyrG markers of Aspergillus nidulans or Aspergillus oryzae and the bar marker of Streptomyces hygroscopicus. Furthermore, selection may be accomplished by co-transformation, e.g., as described in WO 91/17243, where the selectable marker is on a separate vector.

The vectors of the present invention preferably contain an element(s) that permits stable integration of the vector into the host cell genome or autonomous replication of the vector in the cell independent of the genome of the cell.

The vectors of the present invention may be integrated into the host cell genome when introduced into a host cell. For integration, the vector may rely on the nucleotide sequence encoding the polypeptide or any other element of the vector for stable integration of the vector into the genome by homologous or nonhomologous recombination. Alternatively, the vector may contain additional nucleotide sequences for directing integration by homologous recombination into the genome of the host cell. The additional nucleotide sequences enable the vector to be integrated into the host cell genome at a precise location(s) in the chromosome(s). To increase the likelihood of integration at a precise location, the integrational elements should preferably contain a sufficient number of nucleotides, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination. These nucleotide sequences may be any sequence that is homologous with a target sequence in the genome of the host cell, and, furthermore, may be non-encoding or encoding sequences.

For autonomous replication, the vector may further comprise an origin of replication enabling the vector to replicate autonomously in the host cell in question. Examples of bacterial origins of replication are the origins of replication of plasmids pBR322, pUC19, pACYC177, pACYC184, pUB110, pE194, pTA1060, and pAMβ1. Examples of origin of replications for use in a yeast host cell are the 2 micron origin of replication, the combination of CEN6 and ARS4, and the combination of CEN3 and ARS1. The origin of replication may be one having a mutation which makes its functioning temperature-sensitive in the host cell (see, e.g., Ehrlich, 1978, Proceedings of the National Academy of Sciences USA 75:1433).

More than one copy of a nucleotide sequence encoding a polypeptide of the present invention may be inserted into the host cell to amplify expression of the nucleotide sequence. Stable amplification of the nucleotide sequence can be obtained by integrating at least one additional copy of the sequence into the host cell genome using methods well known in the art and selecting for transformants.

The procedures used to ligate the elements described above to construct the recombinant expression vectors of the present invention are well known to one skilled in the art (see, e.g., Sambrook et al., 1989, supra).

### Host Cell Comprising Nucleotide Constructs:

The present invention also relates to recombinant host cells, comprising a nucleotide sequence or nucleotide construct or recombinant expression vector of the invention, which are advantageously used in the recombinant production of the polypeptide variants of the invention. The term “host cell” encompasses a parent host cell and any progeny thereof, which is not identical to the parent host cell due to mutations that occur during replication. The cell is preferably transformed with a vector comprising a nucleotide sequence of the invention followed by integration oft the vector into the host chromosome. “Transformation” means introducing a vector comprising a nucleotide sequence of the present invention into a host cell so that the vector is maintained as a chromosomal integrant or as a self-replicating extra-chromosomal vector. Integration is generally considered to be an advantage as the nucleotide sequence is more likely to be stably maintained in the cell. Integration of the vector into the host chromosome may occur by homologous or non-homologous recombination as described above.

The choice of a host cell will to a large extent depend upon the gene encoding the polypeptide and its source. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulans, Bacillus coagulans, Bacillus lautus, Bacillus lentus, Bacillus lichenifonnis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus lichenifonnis, Bacillus stearothermophilus or Bacillus subtilis cell. The transformation of a bacterial host cell may, for instance, be effected by protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Koehler and Thorne, 1987, Journal of Bacteriology 169:5771-5278).

The host cell may be a eukaryote, such as a mammalian cell, an insect cell, a plant cell or a fungal cell.

Useful mammalian cells include Chinese hamster ovary (CHO) cells, HeLa cells, baby hamster kidney (BHK) cells, COS cells, or any number of other immortalized cell lines available, e.g., from the American Type Culture Collection.

Examples of suitable mammalian cell lines are the COS (ATCC CRL 1650 and 1651), BHK (ATCC CRL 1632,10314 and 1573, ATCC CCL 10), CHL (ATCC CCL39) or CHO (ATCC CCL 61) cell lines. Methods of transfecting mammalian cells and expressing DNA sequences introduced in the cells are described in e.g., Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Ausubel et al., Current Protocols in Molecular Biology, John Wiley and Sons, Inc., N.Y., 1987, Hawley-Nelson et al., Focus 15 (1993), 73; Ciccarone et al., Focus 15 (1993), 80; Graham and van der Eb, Virology 52 (1973), 456; and Neumann et al., EMBO J. 1 (1982), 841-845.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed above. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sorobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9,1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981).

The yeast host cell may be selected from a cell of a species of Candida, Kluyveromyces, Saccharomyces, Schizosaccharomyces, Candida, Pichia, Hansehula, or Yarrowia. In a preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. Other useful yeast host cells are a Kluyveromyces lactis Kluyveromyces fragilis Hansehula polymorpha, Pichia pastoris Yarrowia lipolytica, Schizosaccharomyces pombe, Ustilgo maylis, Candida maltose, Pichia guillermondii and Pichia methanolio cell (cf. Gleeson et al., J. Gen. Microbiol. 132, 1986, pp. 3459-3465; U.S. Pat. No. 4,882,279 and U.S. Pat. No. 4,879,231).

In a preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a vegetative mycelium composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma or a teleomorph or synonym thereof. In an even more preferred embodiment, the filamentous fungal host cell is an Aspergillus cell. In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell. In a most preferred embodiment, the filamentous fungal host cell is an Aspergillus awamori, Aspergillus foetidus, Aspergillus japonicus, Aspergillus niger, Aspergillus nidulans or Aspergillus oryzae cell. In another most preferred embodiment, the filamentous fungal host cell is a Fusarium cell of the section Discolor (also known as the section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, or Fusarium trichothecioides cell. In another preferred embodiment, the filamentous fungal parent cell is a Fusarium strain of the section Elegans, e.g., Fusarium oxysporum. In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell or a Acremonium chrysogenum cell. In another most preferred embodiment, the Trichoderma cell is a Trichoderma harzianum, Trichoderma koningii, Trichoderma longibrachiatum, Trichoderma reesei or Trichoderma viride cell. The use of Aspergillus spp. for the expression of proteins is described in, e.g., EP 272 277, EP 230 023.

The nucleotide sequences, DNA, of the invention may be modified such as to optimize the codon usage for a preferred particular host organism in which it will be expressed. Examples of this are published for yeast (Woo J H, et al, Protein Expression and Purification, Vol. 25 (2), pp. 270-282, 2002), fungi (Te'o et al, FEMS Microbiology Letters, Vol. 190 (1) pp. 13-19 (2000)), and other microorganisms, as well as for Der p 1 expressed in mammalian cells (Massaer M, et al, International Archives of Allergy and Immunology, Vol. 125 (1), pp. 32-43, 2001).

In a particular embodiment the host cell is an insect cell and/or insect cell line. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

### Methods of Preparing Vaccination Antigen Polypeptide Variants

The polypeptide variants of the invention may be prepared by (a) transforming a suitable host cell with a nucleotide construct of the invention, (b) cultivating the recombinant host cell of the invention comprising a nucleotide construct of the invention under conditions conducive for production of the variant of the invention and (c) recovering the variant. The method may in a particular embodiment be carried out as described in WO 01/29078 (HESKA) describing recombinant expression of group 1 mite proteins including nucleotide sequences modified to enable expression of the polypeptides in microorganisms.

### Transformation

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81:1470-1474. A suitable method of transforming Fusarium species is described by Malardier et al., 1989, Gene 78:147-156 or in copending U.S. Ser. No. 08/269,449. Examples of other fungal cells are cells of filamentous fungi, e.g., Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277 and EP 230 023. The transformation of F. oxysporum may, for instance, be carried out as described by Malardier et al., 1989, Gene 78:147-156.

Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; and Hinnen et al., 1978, Proceedings of the National Academy of Sciences USA 75:1920. Mammalian cells may be transformed by direct uptake using the calcium phosphate precipitation method of Graham and Van der Eb (1978, Virology 52:546).

Transformation of insect cells and production of heterologous polypeptides therein may be performed as described in U.S. Pat. No. 4,745,051; U.S. Pat. No. 4,775,624; U.S. Pat. No. 4,879,236; U.S. Pat. No. 5,155,037; U.S. Pat. No. 5,162,222; EP 397,485) all of which are incorporated herein by reference.

### Cultivation

The transformed or transfected host cells described above are cultured in a suitable nutrient medium under conditions permitting the production of the desired molecules, after which these are recovered from the cells, or the culture broth.

The medium used to culture the cells may be any conventional medium suitable for growing the host cells, such as minimal or complex media containing appropriate supplements. Suitable media are available from commercial suppliers or may be prepared according to published recipes (e.g., in catalogues of the American Type Culture Collection). The media are prepared using procedures known in the art (see, e.g., references for bacteria and yeast; Bennett, J. W. and LaSure, L., editors, More Gene Manipulations in Fungi, Academic Press, CA, 1991).

### Recovery

In a particular embodiment the polypeptide variant of the invention is in an isolated and purified form. Thus the polypeptide variant of the invention is provided in a preparation which more than 20% w/w pure, particularly more than 50% w/w pure, more particularly more than 75% W/W pure, more particularly more than 90% w/w pure and even more particularly more than 95% w/w pure. The purity in this context is to be understood as the amount of polypeptide variant of the invention present in the preparation of the total polypeptide material in the preparation.

When applied to a polypeptide, the term “isolated” indicates that the polypeptide is found in a condition other than its native environment, such as apart from blood and animal tissue. In a preferred form, the isolated polypeptide is substantially free of other proteins, particularly other proteins of animal origin. It is preferred to provide the polypeptides in a highly purified form, i.e., greater than 95% pure, more preferably greater than 99% pure.

If the molecules are secreted into the nutrient medium, they can be recovered directly from the medium. If they are not secreted, they can be recovered from cell lysates. The molecules are recovered from the culture medium by conventional procedures including separating the host cells from the medium by centrifugation or filtration, precipitating the proteinaceous components of the supernatant or filtrate by means of a salt, e.g., ammonium sulphate. The proteins may be matured in vitro e.g., by acidification to induce autocatalytic processing (Jacquet et al., Clin Exp Allergy, 2002, vol. 32 pp 1048-53), and they may be purified by a variety of chromatographic procedures, e.g., ion exchange chromatography, gel filtration chromatography, affinity chromatography, or the like, dependent on the type of molecule in question (see, e.g., Protein Purification, J-C Janson and Lars Ryden, editors, VCH Publishers, New York, 1989).

### Compositions

The present invention also relates to a composition comprising a variant of the invention and optionally a pharmaceutically acceptable carrier and/or adjuvant and a method for preparing such a composition comprising admixing the variant of the invention with an acceptable pharmaceutical carrier and/or adjuvant. In a particular embodiment the composition is suitable for treating an immunological disorder, such as allergy in animals or humans, such as a vaccine.

Pharmaceutical carriers and/or adjuvants includes saline, glycerol, aluminium hydroxide, aluminium phosphate, calcium phosphate, saponins (e.g., Q21 and Quill A), squalene based emulsions (e.g., MF59), monophosphoryl lipid A (and synthetic mimics), polylactide co-glycolid (PLG) particles, ISCOMS, liposomes, chitosan, bacterial DNA (e.g., unmethylated CpG containing sequences). Suitable carriers also include pharmaceutically acceptable solvents and/or tabletting aids/auxilliaries.

### Use of Vaccination Antigen Polypeptide Variants and Compositions Containing Them

In a further aspect the invention provide use of the variant or the composition of the invention as a medicament, particularly for the treatment of an immunological disorder, such as allergy in animals and humans and/or for the preparation of a medicament for the treatment of such immunological disorder.

Traditionally, allergy vaccination is performed by parenteral, intranasal, or sublingual administration in increasing doses over a fairly long period of time, and results in, so called, desensitization of the patient. The exact immunological mechanism is not known, but induced differences in the phenotype of allergen specific T and B cells are thought to be of particular importance.

Compared to conventional types of vaccination, allergy vaccination is complicated by the existence of an ongoing immune response in allergic patients. This immune response is characterised by the presence of allergen specific IgE, that will mediate the release of allergic mediators, thereby inducing allergic symptoms upon exposure to allergens. Thus, allergy vaccination using native and/or naturally occurring allergens has an inherent risk of side effects being in the utmost consequence life threatening to the patient.

Approaches to circumvent this problem may be divided in three categories. In practise measures from more than one category are often combined. First category of measures includes the administration of several small and increasing doses over a long period to reach a substantial accumulated dose. The theory being, that the protective immune response slowly is allowed to be initiated, before potentially anaphylactic doses of allergen is administrated. Second category of measures includes physical modification of the allergen by incorporation of the allergen into e.g., a gel formulation such as a aluminium hydroxide. Aluminium hydroxide has an adjuvant effect and a depot effect of slow allergen release, thus reducing the tissue concentration of the allergen. Third category of measures include as described herein modification of the allergen for the purpose of reducing allergenicity.

The immunotherapeutic effect of an allergy vaccine can be assessed in a number of different ways. One is to measure the specific IgE binding, the reduction of which indicates a better safety profile (however not necessarily a better vaccine potential) (WO 99/47680, ALK-ABELLÓ). Also, several cellular assays could be employed to show the modified immuneresponse indicative of good allergy vaccine potential as shown in several publications, all of which are hereby incorporated by reference (van Neerven et al., “T lymphocyte responses to allergens: Epitope-specificity and clinical relevance”, Immunol Today, 1996, vol. 17, pp. 526-532; Hoffmann et al., Allergy, 1999, vol. 54, pp. 446-454, WO99/07880). Basophil histamine release: e.g., Swoboda et al., Eur. J. Immunol., vol. 32, pp 270-280, 2002.

Also skin prick testing could be employed for example as described in Kronqvist et al Clin Exp Allergy 2000 vol 30 pp 670-676

Eventually, clinical trials with allergic patients could be employed using cellular or clinical end-point measurements. (Ebner et al., Clin. Exp. All., 1997, vol. 27, pp. 107-1015; Int. Arch. Allergy Immunol., 1999, vol. 119, pp 1-5).

### Use of Antibody Affinity for Selecting Improved Vaccines

An aspect of the present invention relates to a use of antibody affinity towards a target antigen for selecting one or more immunotherapeutic preparation, comprising a vaccination antigen, having improved properties as a vaccine against the target antigen. In particular the use is characterized by testing antibodies in serum from an animal immunized with the preparation for antibody affinity towards the target antigen and resulting in selection of one or more immunotherapeutic preparation producing antibodies having higher affinity towards the target antigen than at least one preparation tested. Preferably, the antibody is an immunoglobulin.

### EXAMPLES

### Example 1

### Patient Enrolment, Fractionation and Purification of Various Antibody Classes from Serum

In the present work the binding to Bet v 1 of human antibodies from two groups of patients allergic to birch pollen was investigated in relation to other patient characteristics. Ten of the patients had received specific allergy vaccination with Bet v 1 extracts for at least 1.5 year whereas ten other patients were only pharmacologically treated for their symptoms.

There were no statistical differences between the two groups in relation to gender, age and the duration of their birch pollen allergy. One of the patients in the group of ten patients treated with SIT reported no effect of the treatment whereas the other nine had felt a decrease in their allergic symptoms.

Clinical and Paraclinical Tests

Table 1 shows the result of different clinical and paraclinical tests between the SIT-treated group and the non-SIT group. These tests (except for the IgG 1/IgG4 measurements) were carried out at medical centers and were reported with samples. Total IgE and Bet v 1-specific IgE is higher in the SIT group, however the difference is not statistically significant, and the same is the case for the wheal diameter in a skin prick test to Betula verrucosa extracts. However, the allergic symptoms reported by the patients on a visual analogue scale (VAS2002) vary in the two groups; there is a statistical difference with the SIT-treated having less symptoms than the non-SIT group (the lower VAS2002 the less symptoms the patients have).

For patient number 8 the SIT had no effect and the VAS2002 value on 75 was the highest in the SIT-group (Table 2). The amount of Bet v 1-specific IgG1 and Bet v 1-specific IgG4 are higher in the SIT group but only for Bet v 1-specific IgG4 the difference is statistically significant.

TABLE 1
Results of clinical and paraclinical tests, **Mann Whitney test

| | SIT | no SIT | |
|---|---|---|---|
| | N = 10 | N = 10 | P-value** |
| Total IgE, median (kU/l) (range) | 136 (34-398) | 92 (40-365) | 0.0653 |
| Specific IgE median (kU/l) (range) | 41.1 (17.7-76.4) | 22.6 (2.45-77.4) | 0.1306 |
| SPT diameter median (mm) (range) | 7.0 (4-14) | 6.75 (4-9.5) | 0.2897 |
| VAS2002 median (mm) (range) | 26 (1-75) | 59.5 (25-82) | 0.0413 |
| Normalised specific IgG1 median (range) | 0.31 (0.07-1) | 0.19 (0.05-0.75) | 0.5453 |
| Normalised specific IgG4 median (range) | 0.61 (0.21-1) | 0.33 (0.05-0.67) | 0.0355 |

Purification of Total IgE and IgG from Serum

Human IgE and IgG was purified by affinity chromatography from the 21 individual human sera (20 birch sera and 1 lipase serum) from the enrolled patients using an ÄKTAexplorer system (Amersham Biosciences, UK). 5 mg of mouse anti-human-IgE (Novo Nordisk, Denmark) was coupled to 1.5 g sepharose 4B (Amersham Biosciences, UK) following the instructions of the manufacturer. 10 ml serum with NaCl at a final concentration of 0.5 M was applied to the affinity column, followed by equilibration with 100 mM phosphate buffer, pH 8.0 containing 0.5 M NaCl. Bound human IgE was eluted with 0.1 M citric acid. Fractions containing IgE were pooled and concentrated to ˜2 ml using Ultracel Amicon YM30 Ultrafiltration Discs (Millipore, Mass.) while buffer was changed to 100 mM phosphate, pH 8.0.

The effluent from the anti-IgE column was applied on a protein A column (Amersham Biosciences, UK) to purify of total IgG. The column was equilibrated with 100 mM phosphate buffer, pH 8.0, 0.5 M NaCl, and the bound human IgG was eluted with 0.1 M citric acid. Fractions containing IgG were pooled and concentrated to ˜25 ml as described for IgE.

Purification of Bet v 1 Specific IgG from Total IgG

1 mg Bet v 1 was coupled to 1.5 g sepharose 4B (Amersham Biosciences, UK) by following the instructions of the manufacturer. Protein A-purified total IgG was applied on the Bet v 1-affinity column in 100 mM phosphate buffer, pH 8.0, containing 0.5 M NaCl. Subsequently, washing with 100 mM phosphate buffer, pH 8.0 was performed, and bound Bet v 1-specific IgG was eluted with 0.1 M citric acid. Fractions containing protein were neutralised by addition of 0.1 M borate buffer, pH 10.0 and pooled. The buffer was changed to 100 mM phosphate, pH 8.0 and the antibodies were concentrated to ˜3 ml using Ultracel Amicon YM30 Ultrafiltration Discs (Millipore, Mass.).

Purification of Bet v 1 Specific IgG1 and lgG4

For purification of Bet v 1-specific IgG1 and IgG4, M-280 Tosylactivated Dynabeads (Dynal, Norway) were coated with mouse-anti-human-IgG1 and -IgG4 (Biogenesis, UK), respectively. Dynabeads coated with anti-human-IgG4 were added to Bet v 1-specific IgG in 100 mM phosphate, pH 8.0 and incubated for 30 min at room temperature with slow tilt rotation, and washed 5 times with PBS. The Bet v 1-specific IgG4 was eluted with 400 μl 100 mM citric acid, which was neutralised with 1.6 ml 0.1 M borate, pH 10.0. Dynabeads were washed 4 times in PBS and reused.

Subsequently, Dynabeads coated with anti-human-IgG1 were used for isolation of Bet v 1-specific IgG1 from IgG4-depleted Bet v 1-specific IgG. The eluted antibodies were concentrated to ˜500 μl and the buffer was changed to PBS, pH 7.4 using Centricon YM-10 Centrifugal Filter Unit (Millipore, Mass.).

### Western Blotting

Purified antibodies were analysed by western blotting. The various fractions were separated on 14% SDS-polyacrylamide gels (Invitrogen, CA) under reducing conditions (Sambrook et al., Molecular cloning: A laboratory manual. Cold Spring Harbor, N.Y.: Cold Spring Harbor Laboratory Press, 1989) and transferred to PVDF membranes (Invitrogen, CA) (Laemmli, Nature, 227, 680-685, 1970). After blotting the membranes were blocked overnight in 5% (w/v) skim milk powder in PBS.

IgG1 and IgG4 were detected with mouse-anti-human-IgG1 (Biogenesis, UK) or mouse anti-human-IgG4 (Biogenesis, UK) diluted 1:1000, respectively. Antigen-antibody complexes were detected with rabbit-anti-mouse-Ig (DakoCytomation, Denmark) diluted 1:2000, and HRP-conjugated goat-anti-rabbit-Ig (DakoCytomation, Denmark) diluted 1:2000. IgE was detected using rabbit-anti-human-IgE (DakoCytomation, Denmark) diluted 1:1000, goat-anti-rabbit-Ig conjugated with biotin (DakoCytomation, Denmark) diluted 1:5000, and HRP-conjugated streptavidin (KPL, Maryland, USA) diluted 1:1000. Membranes were washed 3 times in PBS supplemented with 0.1% Tween20 between each incubation. Blots were developed using DAB (Sigma-Aldrich, Mo.).

Results from Purification of Human Antibodies

Total human IgE and Bet v 1 specific IgG1 and IgG4 were purified from only 10 ml serum from each of 20 patients allergic to birch pollen. First 10 ml serum was applied on an anti-human-IgE column to purify total IgE. The effluent from this column was applied on a protein A column to purify total IgG. The fractions containing IgG were then loaded on a Bet v 1-column to achieve Bet v 1-specific IgG. To the positive fractions magnetic beads coated with either anti-human -IgG1 or anti-human-IgG4 were added to purify Bet v 1-specific IgG1 and -IgG4.

A Western blot was run with the purified IgE, Bet v 1-specific IgG1 and Bet v 1-specific IgG4 from two patients plus controls with commercial IgE, IgG1, and IgG4. IgE was detected with anti-human-IgE whereas IgG1 and IgG4 were detected by anti-human-IgG1/IgG4.

One western blot of the purified antibodies from two of the patients shows a band at about 70 kDa corresponding to the heavy chain of the human IgE which is seen in all three lanes; the first lane is a commercial human IgE and the following two lanes are the purified IgE from patient number 12 and 16. The amount of IgE purified from 10 m I serum from each patient varied from 40 ng-14.5 μg (measured by ELISA).

Another western blot shows the purified Bet v 1-specific IgG1 and IgG4 from patient number 12 and 16 together with a commercial IgG1 and IgG4. For IgG1 only the commercial IgG1 gave rise to a band at 50 kDa corresponding to the human IgG heavy chain as the concentration of the purified Bet v 1-specific IgG1 is too low to be seen on a western blot. However, it was still possible to measure the amount of IgG1 by ELISA, where the amount of IgG1 purified from 10 ml serum varied from 3-350 ng between the 20 patients. By ELISA small impurities of IgG4 in the purified IgG1 were observed, but though the anti-human-IgG4 from the manufacturer was reported not to react with other human IgG subclasses, some cross-reaction with IgG1 has been observed previously. The commercial IgG4 and the purified Bet v 1-specific IgG4 gave rise to a band at 50 kDa also corresponding to the heavy chain. No IgG1 impurities were observed. The amount of purified IgG4 from each patient serum sample varied from 10-600 ng.

### Example 2

### Measurement of Anti-Serum Apparent Affinity

### Materials and Methods:

### Determination of the Concentrations of the Purified Antibodies

To measure the concentration of the purified antibodies different ELISAs were used.

IgE was measured by coating an ELISA maxisorp plate (Nunc, Denmark) with mouse-anti-human-IgE (Zymed, CA) diluted 1:4000 overnight at 4° C. After blocking with 2% (w/v) skim milk powder in PBS, IgE purified from the 20 serum samples diluted 1:10 and a known standard of human IgE (Biogenesis, UK) was added in different concentrations. The bound IgE was detected with HRP-conjugated goat-anti-human-IgE (Serotec, UK) diluted 1:3200. Washing and staining was performed as described above.

IgG1 and IgG4 was measured by coating an ELISA maxisorp plate (Nunc, Denmark) overnight at 4° C. with purified IgG1 or IgG4 diluted 1:10 and a known standard of human IgG1 (Serotec, UK) and IgG4 (Nordic Immunologicals, The Netherlands), respectively. After blocking with 2% (w/v) skim milk powder in PBS, the bound IgG1 or IgG4 was detected with mouse-anti-human-IgG1 (Biogenesis, UK) or mouse anti-human-IgG4 (Biogenesis, UK) diluted 1:1000, biotin-conjugated rat-anti-mouse-IgG1 diluted 1:2000 (DakoCytomation, Denmark), and HRP-conjugated streptavidin (KPL, Maryland, USA) diluted 1:1000. Washing and staining was performed as described above.

Surface Plasmon Resonance (Biacore) with Purified Antibodies

Recombinant Bet v 1 was immobilised on a CM5 biosensor chip at a concentration of 15 μg/ml in 10 mM sodium acetate, pH 4.0 using the N-hydroxy-succinimide (NHS)/N-ethyl-N′-(3-dimethylaminopropyl) carbodiimide hydrochloride (EDC) kit (Biacore, Sweden), yielding a surface of ˜1000 RU. Purified antibodies were injected non-diluted or 2-fold diluted in HBS-EP running buffer (10 mM HEPES, 3.4 mM EDTA, 150 mM NaCl), 0.05% (v/v) surfactant P20, pH 7.4). Serum samples were injected 20-fold diluted in HPS-EP running buffer, and antibodies binding to Bet v 1 were analysed using a flow rate of 4 μl/min. The kinetics of binding was analysed using the BIAevaluation software.

### Statistics

All data were analysed using the statistical software program GraphPad InStat version 3.0. The baseline characteristics of the SIT and the non-SIT group were compared using Fisher's exact test for categorical variables and by the Mann-Whitney U-test for continuous variables. The Mann-Whitney U-test was also used for comparison between the two groups of all the obtained data. The Spearman test for correlations was used for comparison of VAS and the binding affinities.

### Results

### Analysis of the Antibody-Bet v 1 Interaction

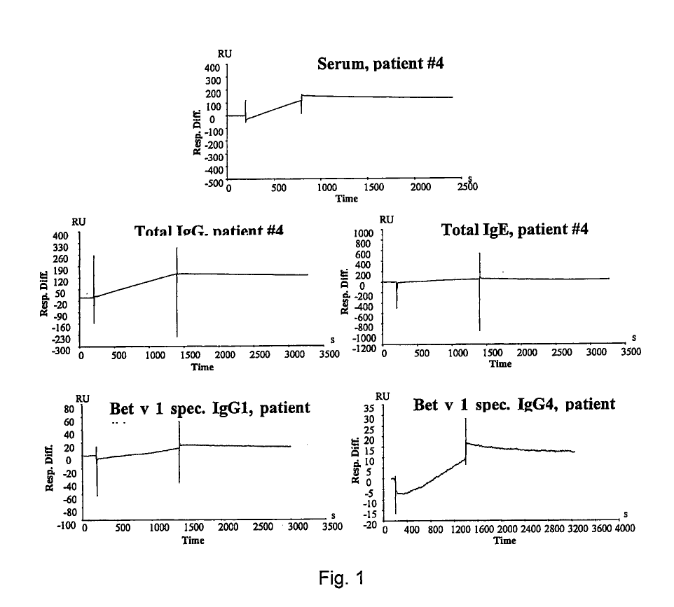

The binding affinities (Kₐ) to Bet v 1 were measured by surface plasmon resonance. FIG. 1 shows a typical sensorgram for sera or purified antibodies binding to Bet v 1 on a chip. The on rate, kₒₙ, of the binding is determined from the first part of the curve where the antibody is injected and the off rate, k_{off}, is determined from the second part of the curve where the bound antibodies dissociate again. From the kₒₙ and k_{off} the binding affinity, Kₐ is calculated. This value is an apparent (“average”) binding affinity as the injected antibodies come from a polyclonal mixture of antibodies with different affinities to Bet v 1 and since the antibody binding interaction is bivalent.

Table 2 shows the apparent binding affinity to Bet v 1 of total IgG, Bet v 1-specific IgE, -IgG1, and -IgG4. The binding affinities of the purified total IgG range between 10⁶-10⁸ M⁻¹, whereas the affinities for the specific antibodies are in a higher range. The concentration of specific IgE used for calculation of the Kₐ was based on the percentage of specific IgE in relation to total IgE (Table 1). Bet v 1-specific IgE have affinities at ˜10⁹-10¹¹ M⁻¹, which is the same range as for Bet v 1-specific IgG1. Bet v 1-specific IgG4 have affinities in the range ˜10⁸-10¹¹M⁻¹.

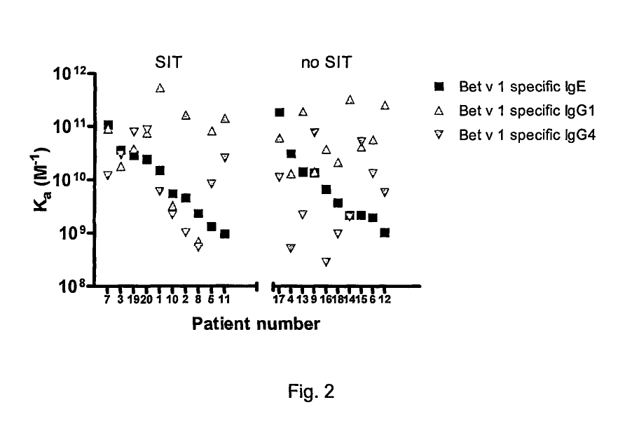

The Kₐ-values for the individual patients are plotted in FIG. 2. For 10 out of 20 patients, the Kₐ for IgG1 has the highest value, and for 4 out of 20 patients, IgG4 has the highest affinity.

Table 2 and FIG. 1 show the maximal amount, Rₘₐₓ, of antibodies in serum diluted 20 times that binds to a chip with immobilised Bet v 1. All patients in the SIT group have a higher Rₘₐₓ value than the non-SIT group. Table 3 shows that the difference is statistically very significant. The R^{max} reflects binding of Bet v 1-specific antibodies of all isotypes. It would have been interesting with blood samples before SIT-treatment from the SIT group to make sure it was identical to the non-SIT group, as the patients in the SIT group might have been chosen for SIT because of severe symptoms leading to a bias compared to the non-SIT group. Patient number 8, for which the SIT did not have an effect, has the highest Rₘₐₓ value.

Table 3 shows the median of the different on and off rates plus the binding affinities. It is seen that there are no statistical significant differences between the SIT and the non-SIT group for either total IgG, Bet v 1-specific IgE, -IgG1, or -IgG4.

TABLE 2
Surface plasmon resonance results plus symptom scores (VAS2002) for the
individual patients. Apparent binding affinity Kₐ (M⁻¹) = kₒₙ/k_{off} for
the antibodies purified from persons allergic to birch pollen. The value is an average
of the affinities of the polyclonal antibodies in the samples. All affinities are a result of at
least two experiments.

| | | | Bet v 1- | Bet v 1- | Bet v 1- | |
|---|---|---|---|---|---|---|
| | Rₘₐₓ | | specific | specific | specific | |
| | serum | Total IgG | IgE | IgG1 | IgG4 | VAS2002 |
| | (RU) | Ka (M⁻¹) | Ka (M⁻¹) | Ka (M⁻¹) | Ka (M⁻¹) | (mm) |
| SIT | | | | | | |
| Patient #1 | 2170 | 1.90 × 10⁶ | 1.50 × 10¹⁰ | 5.40 × 10¹¹ | 5.89 × 10⁹ | 11 |
| Patient #2 | 1210 | 7.19 × 10⁵ | 4.56 × 10⁹ | 1.56 × 10¹¹ | 1.48 × 10⁹ | 53 |
| Patient #3 | 962 | 8.87 × 10⁶ | 3.59 × 10¹⁰ | 1.81 × 10¹⁰ | 4.73 × 10⁹ | 35 |
| Patient #5 | 3450 | 2.05 × 10⁷ | 1.32 × 10⁹ | 8.27 × 10¹⁰ | 8.26 × 10⁹ | 32 |
| Patient #7 | 1710 | 3.09 × 10⁷ | 1.07 × 10¹¹ | 9.01 × 10¹⁰ | 1.15 × 10¹⁰ | 17 |
| Patient #8* | 4680 | 1.72 × 10⁷ | 2.33 × 10⁹ | 7.00 × 10⁸ | 7.72 × 10⁸ | 75 |
| Patient #10 | 3320 | 1.22 × 10⁷ | 5.51 × 10⁹ | 3.29 × 10⁹ | 2.22 × 10⁹ | 66 |
| Patient #11 | 2770 | 5.84 × 10⁶ | 9.58 × 10⁸ | 1.48 × 10¹⁰ | 2.45 × 10¹⁰ | 1 |
| Patient #19 | 1120 | 2.56 × 10⁷ | 2.88 × 10¹⁰ | 1.12 × 10¹⁰ | 1.03 × 10¹¹ | 9 |
| Patient #20 | 679 | 8.95 × 10⁶ | 2.40 × 10¹⁰ | 1.14 × 10¹¹ | 8.60 × 10¹⁰ | 20 |
| No SIT | | | | | | |
| Patient #4 | 317 | 3.18 × 10⁶ | 3.03 × 10¹⁰ | 1.94 × 10¹⁰ | 1.91 × 10⁸ | 58 |
| Patient #6 | 25 | 6.27 × 10⁷ | 1.95 × 10⁹ | 5.58 × 10¹⁰ | 1.26 × 10¹⁰ | 79 |
| Patient #9 | 0 | 3.19 × 10⁷ | 1.35 × 10¹⁰ | 1.37 × 10¹⁰ | 7.51 × 10¹⁰ | 65 |
| Patient #12 | 339 | 2.58 × 10⁶ | 1.01 × 10⁹ | 2.52 × 10¹¹ | 5.69 × 10⁹ | 55 |
| Patient #13 | 27 | 5.13 × 10⁷ | 1.41 × 10¹⁰ | 1.90 × 10¹¹ | 2.20 × 10⁹ | 66 |
| Patient #14 | 5 | 4.51 × 10⁷ | 2.16 × 10⁹ | 3.15 × 10¹¹ | 1.97 × 10⁹ | 25 |
| Patient #15 | 427 | 1.45 × 10⁷ | 2.16 × 10⁹ | 4.13 × 10¹⁰ | 5.05 × 10¹⁰ | 28 |
| Patient #16 | 241 | 8.92 × 10⁷ | 6.64 × 10⁹ | 3.72 × 10¹⁰ | 2.82 × 10⁸ | 82 |
| Patient #17 | 0 | 1.87 × 10⁷ | 1.82 × 10¹¹ | 6.07 × 10¹⁰ | 1.11 × 10¹⁰ | 37 |
| Patient #18 | 493 | 3.07 × 10⁷ | 3.70 × 10⁹ | 2.08 × 10¹⁰ | 9.63 × 10⁸ | 61 |

RU: resonance units.
*No effect of immunotherapy.

TABLE 3
Surface plasmon resonance results for the SIT and non-SIT groups. On and off rates
for the binding to Bet v 1 of antibodies purified from persons allergic to birch pollen were used
to calculate the apparent binding affinity Kₐ (M⁻¹) = kₒₙ/k_{off}. The values are an average of the
affinities of the polyclonal antibodies in the samples

| | SIT | no SIT | |
|---|---|---|---|
| | N = 10 | N = 10 | P-value** |
| Rmax (serum) median/RU | 1940 | 134.05 | <0.0001 |
| (range) | (679-4680) | (0-493) | |
| kon (IgE) median/s⁻¹ | 4.07 × 10⁵ | 5.91 × 10⁵ | 0.7394 |
| (range) | (7.32 × 10⁴-2.38 × 10⁷) | (5.52 × 10⁴-2.46 × 10⁷) | |
| koff (IgE) median/M⁻¹ · s⁻¹ | 9.60 × 10⁻⁵ | 1.82 × 10⁻⁴ | 0.4813 |
| (range) | (9.18 × 10⁻⁶-3.21 × 10⁻⁴) | (6.94 × 10⁻⁶-1.75 × 10⁻³) | |
| Ka (IgE) median/M⁻¹ | 1.03 × 10¹⁰ | 5.17 × 10⁹ | 0.6305 |
| (range) | (9.58 × 10⁸-1.07 × 10¹¹) | (1.01 × 10⁹-1.82 × 10¹¹) | |
| kon (IgG1) median/s⁻¹ | 2.36 × 10⁷ | 3.35 × 10⁷ | 0.6842 |
| (range) | (1.55 × 10⁶-1.07 × 10⁸) | (1.74 × 10⁶-1.13 × 10⁸) | |
| koff (IgG1) median/ | 5.19 × 10⁻⁴ | 4.49 × 10⁻⁴ | |
| M⁻¹ · s⁻¹ | (1.98 × 10⁻⁴-4.04 × 10⁻³) | (8.61 × 10⁻⁵-9.7 × 10⁻⁴) | 0.6842 |
| (range) | | | |
| Ka (IgG1) median/M⁻¹ | 5.04 × 10¹⁰ | 4.86 × 10¹⁰ | 0.4813 |
| (range) | (7.00 × 10⁸-5.40 × 10¹¹) | (1.37 × 10¹⁰-3.2 × 10¹¹) | |
| kon (IgG4) median/s⁻¹ | 1.00 × 10⁷ | 1.97 × 10⁶ | 0.3930 |
| (range) | (1.02 × 10⁵-2.97 × 10⁷) | (4.26 × 10⁴-4.15 × 10⁷) | |
| koff (IgG4) median/ | 1.11 × 10⁻³ | 5.63 × 10⁻⁴ | 0.1051 |
| M⁻¹ · s⁻¹ | | | |
| (range) | (7.21 × 10⁻⁵-1.85 × 10⁻³) | (9.30 × 10⁻⁵-1.04 × 10⁻³) | |
| Ka (IgG4) median/M⁻¹ | 7.08 × 10⁹ | 3.95 × 10⁹ | 0.3930 |
| (range) | (7.72 × 10⁸-1.03 × 10¹¹) | (1.91 × 10⁸-7.51 × 10¹⁰) | |

**Mann Whitney test.

### Affinity in Relation to Symptoms

The affinities to Bet v 1 of total Bet v 1-specific IgE, -IgG1 and -IgG4 for each patient from the tables above can be plotted against the symptoms of the patients (logKa against VAS2002) with the following result:

Between the affinities of the total IgE and the VAS2002 score there is no correlation in any of the groups. For Bet v 1-specific I gG1 there is a correlation showing that the higher antibody affinity the less symptoms. The correlation is not statistically significant for the non-SIT group, but when you look at the SIT group, and all patients combined the correlation is significant. Bet v 1-specific IgG4 shows the same pattern. In the non-SIT group there is no statistical correlation but the Spearman correlation test shows that in the SIT group there is correlation proving that the patients with the lowest symptom scores have the antibodies with the highest affinities, and the same is the case when looking at all patients.

Between the amounts of Bet v 1-specific IgG1 or IgG4 and the VAS2002 score, there was no correlation.

### Example 3

### Finding of Epitope Patterns within Oligo Peptides with Antibody Reactivity

A high diversity library of phages expressing random oligomeric peptides (hexa, hepta, octa, nona and/or dodeca peptides) as part of their surface proteins, were screened for their capacity to bind antibodies. The phage libraries were obtained from Schafer-N, Copenhagen, Denmark.

Antibody samples were raised in animals (Rat, Rabbits or Mice) by parenteral or mucosal administration of each of the proteins listed below. The antibodies were dissolved in phosphate buffered saline (PBS). In some cases, antibodies of specific subclasses were purified from the serum of immunised animals by capryilic acid precipitation (for total IgG) or by affinity chromatography using paramagnetic immunobeads (Dynal AS) loaded with one of the following antibodies: mouse anti-rat IgG1 or rat anti-mouse IgE.
1) amylase AA560 from Bacillus sp. DSM 12649, (Rat IgG)
2) alpha-amylase of Bacillus halmapalus (WO96/23873), which is also called amylase SP722, (Rat IgG)
3) a variant of SP722 with residues 183 and 184 deleted, called JE-1 (WO96/23873), (Rat IgG and Rabbit IgG)
4) Mycelioptora thermopila laccase (WO95/33836) (Rabbit IgG),
5) T. lanuginosus lipase (Lipolase™) (Rat IgG and Rabbit IgG),
6) family 45 cellulase from Humicola insolens (Carezyme™) (Rabbit IgG),
7) Bacillus lentus protease (Savinase™) (Rat IgG, Mouse IgG, Mouse IgE, and Rabbit IgG),
8) Subtilisin Novo (BPN′) from B. amyloliquefaciens (Rat IgG),
9) The Y217L variant of Subtilisin Novo (Rat IgG),
10) Subtilisin Carlsberg (Alcalase™) (Rat IgG),
11) TY145 protease (Rat IgG),
12) CDJ31 protease,
13) Subtilisin 147 (Esperase™) (Rat IgG),
14) Bacillolysin from Bacillus amyloliquefaciens (Neutrase™) (Rat IgG and Rat IgG1), and
15) Subtilisin PD498 (WO93/24623) (Rat IgG and Rabbit IgG).

The phage libraries were incubated with the antibody coated beads. E.g. phages expressing oligo-peptides with affinity for mouse IgE antibodies were captured onto rat anti-mouse IgE-coated beads and collected by exposing these paramagnetic beads to a magnetic field. The collected phages were eluted from the immobilised antibodies by mild acid treatment, or by elution with intact protein antigen specific for the respective antibody sample (e.g., Savinase for anti-Savinase antibodies). The isolated phages were amplified using methods known in the art. Alternatively, immobilised phages were directly incubated with E. coli for infection. In short, F-factor positive E. coli (e.g., XL-1 Blue, JM101, TG1) were infected with M13-derived vector in the presence of a helper phage (e.g., M13K07), and incubated, typically in 2xYT containing glucose or IPTG, and appropriate antibiotics for selection. Finally, cells were removed by centrifugation. This cycle of events was repeated on the respective cell supernatants, minimum 2 times and maximum 5 times. After selection round 2, 3, 4 and 5, a fraction of the infected E. coli was incubated on selective 2xYT agar plates, and the specificity of the emerging phages was assessed immunologically: Phages were transferred to a nitrocellulase (NC) membrane. For each plate, 2 NC-replicas were made. One replica was incubated with the selection antibodies, the other replica was incubated with the selection antibodies and the immunogen used to obtain the antibodies as competitor. Those plaques that were absent in the presence of immunogen, were considered specific, and were amplified according to the procedure described above.

The specific phage-clones were isolated from the cell supernatant by centrifugation in the presence of polyethylenglycol. DNA was isolated, the DNA sequence coding for the oligopeptide was amplified by PCR, and its DNA sequence was determined, all according to standard procedures known in the art. The amino acid sequence of the corresponding oligopeptide was deduced from the DNA sequence.

These 256 experimentally determined reactive peptides were supplemented with information on 402 reactive peptides published in the literature:
Allergy 38 (1983) 449-459,
Allergy 56 (2001) 118-125;
Allergy 56 s67 (2001) 48-51;
Allergy 54 (1999) 1048-1057;
Arch Biochem Biophys 342 (1997) 244-253
B. B. Res. Com. 219 (1996) 290-293;
Biochem J 293 (1993) 625-632;
Bioinformatics 18 (2002) 1358-1364;
Clin Exp Allergy 24 (1994) 100-108;
Clin Exp Allergy 24 (1994) 250-256;
Clin Exp Allergy 31 (2001) 331-341;
Clin Exp Med 24 (1994) 100-108;
Eur J Biochem 245 (1997) 334-339;
Int Arch Allergy Appl Immunol 89 (1989) 342-348
Int Arch Allergy Appl Immunol 89 (1989) 410-415
Int Arch Appl Immunol 103 (1994) 357-364
Int Arch Appl Immunol 92 (1990) 30-38
J Allergy Clin Immunol 106 (2000) 150-158
J Allergy Clin Immunol 107 (2001) 1069-1076
J Allergy Clin Immunol 93 (1994) 3443
J Biol Chem 271 (1996) 29915-29921
J Clin Invest 103 (1999) 535-542
Immunol 121 (1989) 275-280
J Immunol 133 (1984) 2668-2673
J Immunol 140 (1988) 611-616
J Immunol 147 (1991) 205-211
J Immunol 151 (1993) 5354-5363
J Immunol 151 (1993) 7206-7213
J Immunol Methods 213 (1998) 1-17
Mol Immunol 25 (1988) 355-365
Mol Immunol 28 (1991) 1225-1232
Mol Immunol 29 (1992) 1383-1389
Mol Immunol 29 (1992) 257-262
Mol Immunol 30 (1993) 183-189
Mol Immunol 35 (1998) 293-305
Mol Immunol 37 (2000) 789-798
Peptides 21 (2000) 589-599
Protein Science 8 (1999) 760-770
Scand J Immunol 27 (1988) 587-591
Science 233 (1986) 747-753
WO 90/11293
WO 99/38978
WO 01/34186
WO 01/39799
WO 01/39799
WO 01/49834
www.csl.gov.uk/allergen

Thus, in total 658 peptide sequences with specificity for the protein-specific antibodies, described above, were obtained. These sequences were collected in a database, and analysed by sequence alignment to identify epitope patterns observing that conservative alternatives were considered equal (as described above).

### Identifying Epitope Patterns

In principle, each of the 658 reactive (oligo)peptide sequences represented an epitope pattern. However, in the 658 reactive (oligo)peptide sequences some epitope patterns were redundant and to remove redundancy among the epitope patterns, the reactive (oligo)peptides sequences were subjected to computerised data analysis.

First all possible dipeptides were generated corresponding to 13² different combinations taking conservative alternatives into account. The presence and frequency of each dipeptide among the 658 reactive (oligo)peptide sequences were listed. Next all possible tripeptides were generated corresponding to 13³ different combinations and again the presence and frequency of each tripeptide among the 658 reactive (oligo)peptide sequences were listed. All possible combinations of the listed dipeptides and tripeptides were then generated including those containing 1, 2, 3 or 4 residues inserted between the dipeptides and tripeptides, these residues selected among the 13 possible residue types. This procedure generated a list of different peptide combinations of 5 to 9 amino acids each containing at least one dipeptide and at lest one tripeptide from the initial listings as well as 0 to 4 residues in between. The frequency of each peptide combination among the 658 reactive (oligo)peptide sequences were then ranked and relevant epitope patterns were selected by a procedure where reactive peptides covered by the most frequent combination were first selected and separated from the group of the 658 reactive peptides. Then reactive peptides covered by the second most frequent combination were selected and separated from the remaining group. Then reactive peptides covered by the third most frequent combination were selected and separated from the remaining group. This procedure was repeated until combinations covering all 658 reactive peptides are found. This way it was found that 357 combinations (epitope patterns) were found to cover all the 658 reactive peptides.

### Example 4

### Predicting Epitopes

The Der p 1 model was built using the following three-dimensional structures as templates:

| PDB entry | Protein |
|---|---|
| 9PAP | Papaya papain |
| 2ACT | Kiwi actinidin |
| 1PPO | Papaya omega protease |

The sequences of the three templates and mature Der p 1 were aligned using ClustalW 1.7 (as described in Higgins et al., Methods Enzymol., vol. 266, pp 383-402, 1996, and Thompson, et al., Nucleic Acids Research, vol. 22, pp 4673-4680, 1994. As the first 9 residues of mature Der p 1 have no corresponding residues in any of the templates, reliable modelling of these residues is not possible, and hence, the 9 N-terminal residues are not included in the Der p 1 model.

The “Modeler 5.0” program (Accelrys Software, San Diego, Calif., USA) was used to build the three-dimensional model of Der p 1. “Modeler 5.0” was started from the “Insightll” molecular modelling software (Accelrys Software, San Diego, Calif., USA) using the following parameters: Number of models: 1, Optimize level: None, More options: Yes, Optimize loop: Yes, Number of loop models: 2, Loop optimite level: Low, Build hydrogens: None.

Using “Insightll”, hydrogens were added and CHARMm potentials and partial charges assigned to the model. Using “CHARMm” (Accelrys Software, San Diego, Calif., USA), 100 steps of ABNR mimimization were applied to relax the model.

Surface accessibility was measured for each amino acid in Der p 1 (SEQ ID NO:1) using the DSSP program (see W. Kabsch and C. Sander, Biopolymers 22 (1983) 2577-2637) in percent of a standard value for that amino acid. The standard values generated according to established methods by analysing average surface accessibility of an amino acid in a 20-mer homogeneous peptide in helix formation using the DSSP program. For each of the 20 amino acids the average surface accessibility were as follows:

| Residue | Accessiblity Å² |
|---|---|
| A | 62 |
| C | 92 |
| D | 69 |
| E | 156 |
| F | 123 |
| G | 50 |
| H | 130 |
| I | 84 |
| K | 174 |
| L | 97 |
| M | 103 |
| N | 85 |
| P | 67 |
| Q | 127 |
| R | 211 |
| S | 64 |
| T | 80 |
| V | 81 |
| W | 126 |
| Y | 104 |

Epitopes were predicted by a computer program on a 3-dimensional model of Der p 1 by using the epitope patterns found in example 1 as follows:

(1) For all amino acids in Der p 1 it was examined if (a) the amino acid type match the first amino acid of an epitope patterns and (b) the solvent surface accessibility greater than or equal to a predefined value, e.g., 20%. Those amino acid satisfying 1(a) and 1(b) are selected.
(2) For all amino acids within a distance of 10 Å from the amino acids selected in step 1 it is examined if (a) the amino acid type matches the second amino acid of the pattern and (b) the surface accessibility greater than or equal to the predefined value, e.g., 20%. Those amino acid satisfying 2(a) and 2(b) are selected.
(3) For all amino acids within a distance 10 Å from the amino acids selected in step 2 it is examined if (a) the amino acid type matches the third amino acid of the pattern and (b) the surface accessibility greater than or equal to the predefined value, e.g., 20%. Those amino acid satisfying 3(a) and 3(b) are selected.
(4) Repeating step 3 for all amino acids in the epitope pattern

Further, a limit of 25 Å was set as the maximum distance between any two epitope residues.

This procedure was carried out for all 357 epitope patterns for each of the following settings for surface accessibility cutoff: 30, 40, 50, 60, 70 and 80%. Epitope patterns finding a match on the 3 dimensional structure of Der p 1 according this procedure are predicted as epitopes.

Finally, for each of the seven settings for solvent accessibility, a table of all Der p 1 amino acids was created, in which each amino acid residue was given a score by adding up the number of times it appeared in one of the epitopes (at that solvent setting). This score will be an indication of the likelihood that modification (substitution, insertion, deletion, glycosylation or chemical conjugation) of that amino acid will, result in a variant of lower antigenicity. All amino acids of the protein can then be ranked according to this score and those with highest scores can be selected for mutagenesis.

The 10% highest scoring amino acids (i.e. the 22 highest scoring ones), at each solvent accessibility setting, are shown in Table 4 below (however for solvent accessibility settings of 70 and 80%, less than 22 residues scored at all, so in those two cases all residues that scored are listed). Cysteines are omitted, as they are often involved in maintaining three-dimensional structure.

TABLE 4
Highest scoring residues for epitope mapping of Der p 1.

| Minimum | |
|---|---|
| Solvent | |
| Accessibility | Top scoring 10% of amino acids |
| 20% | G32, A46, Y47, L55, D64, A66, S67, G73, T75, I80, Q84, N86, G87, A98, |
| | R105, Q109, R110, F111, G112, I113 |
| 30% | G30, G32, D64, A66, S67, Q84, N86, G87, Y93, A98, R99, Q101, R105, |
| | P106, F111, G112, I113, I158, A205 |
| 40% | G29, G30, G 32, L 55, A 66, S 67, Q84, S 92, Y 93, Y 96, A 98, R 99, E 100, |
| | Q101, R104, Q109, F111, I113, I144, K145, D146, N163 |
| 50% | G29, G30, G32, S67, R99, E100, I144, K145, D146, D148, R151, I159, |
| | Q160, R161, D162, N163, G164, Y165, Q166, A180, V183, D184 |
| 60% | A10, A12, E13, A132, I144, K145, D146, D148, R151, I159, Q160, R161, |
| | D162, N163, G164, Y165, Q166, N179, A180, G182, D184, I208 |
| 70% | A10, A12, I144, K145, D146, D148, I159, R161, G164, Y165, Q166, D184, |
| | I208 |
| 80% | D146, D148, G164, Y165, Q166 |

From this procedure it was found that residues A10, A12, E13, G29, G30, G32, A46, Y47, L55, D64, A66, S67, G73, T75,180, Q84, N86, G87, S92, Y93, Y96, A98, R99, E100, Q101, R104, R105, P106, Q109, R110, F1, G112, I113, A132, I144, K145, D146, D148, R151, I158, I159, Q160, R161, D162, N163, G164, Y165, Q166, N179, A180, G182, V183, D184, A205, I208 of SEQ ID NO:1, each belonged to the top 10% highest ranking residues at least one solvent accessibility setting.

Residues A10, A12, G30, G32, A46, Y47, L55, D64, A66, S67, G87, S92, A98, R99, E100, Q101, R105, R110, F111, G112, I113, I144, K145, D146, D148, R151, I159, Q160, R161, D162, N163, G164, Y165, Q166 of SEQ ID NO:1, each belonged to the top 5% highest ranking residues at least one solvent accessibility setting.

By studying the positions of the top 10% scoring amino acids on the three-dimensional model of Der p 1, it is possible to define 5 epitope containing regions on the molecule. The 50% highest scoring amino acids within each region are then selected as best candidates for mutation: Residues A10, A12, G32, L55, A66, S67, G87, A98, R99, F111, G112, I113, I144, D146, D148, I159, R161, G164, Q166, A180, D184, A205 and I208 of SEQ ID NO:1

1-21. (canceled)

22. A method for selecting one or more immunotherapeutic preparation having improved properties as a vaccine against a target antigen, said method comprising raising antibodies against a group of candidate preparations and selecting one or more preparations which produces antibodies having higher affinity against the target antigen than does antibodies raised against at least one other preparation from the group.

23. The method of claim 22, wherein the preparation(s) each comprises different vaccination antigens.

24. The method of claim 22, wherein the preparation(s) are formulated with different adjuvants.

25. The method of claim 22, wherein the preparation(s) comprise the target antigen subjected to various physical or chemical modification steps.

26. The method of claim 25, wherein the modification is fragmentation.

27. The method of claim 22, wherein the target antigen is an allergen.

28. The method of claim 27, wherein the allergen is an environmental allergen or a commercial allergen.

29. The method of claim 22, comprising the steps:
a) providing two or more vaccine preparations,
b) immunizing animals or groups of animals with one preparation each,
c) measuring target antigen affinity towards serum immunoglobulin isolated from the animals,
d) selecting one or more preparations resulting in higher immunoglobulin affinity towards the target antigen than at least one other preparation.

30. The method of claim 22, wherein the vaccination antigens are pre-selected for affinity towards anti-target antigen antiserum.

31. The method of claim 25, wherein relevant positions for modification in the target antigen is identified by epitope mapping, the target antigen is modified at relevant positions to produce variants, and the variants are included in separate candidate preparations.

32. The method of claim 31, wherein the modification of an identified epitope affect antigenicity.

33. The method of claim 32, wherein the antigenicity of the vaccination antigen is higher compared to the target antigen.

34. A method for selecting one or more improved immunotherapeutic administration regimes for a vaccine against a target antigen, said method comprising raising antibodies against the vaccine through a group of candidate administration regimes and selecting one or more administration regimes having higher affinity against the target antigen than antibodies raised against at least one other administration regime from the group.

35. The method of claim 34, comprising the steps:
a) providing one or more vaccine preparations,
b) immunizing animals or groups of animals, each by different administration regime,
c) measuring target antigen affinity towards serum immunoglobulin isolated from the animals, and
d) selecting one or more administration regimes or combinations of preparation and administration regime resulting in antibodies having higher affinity against the target antigen than antibodies raised against at least one of the tested administration regimes or regimes in combination with preparations.

36. The method of claim 35, wherein the animal is naive or pre-sensitized.

37. The method of claim 35, wherein a sub-fraction of serum immunoglobulin is isolated before step (c).

38. The method of claim 37, wherein the sub-fraction comprises specific IgG, IgG1, and/or IgG4.

39. The method of claim 35, wherein the administration regime comprises intranasal, intratracheal, subcutaneous, or intraperitoneal administration.
